(19) United States
(12) Patent Application Publication
(10) Pub. No.: US 2008/0193484 A1
(43) Pub. Date: Aug. 14, 2008

(54) Novel Methods for Producing Adenoviral Vector Preparations with Reduced Replication-Competent Adenovirus Contamination and Novel Adenoviral Vectors and Preparations

(75) Inventors: Xinzhong Wang, Framingham, MA (US); George C. Kaynor, Melrose, MA (US); James Barsoum, Lexington, MA (US)

Correspondence Address: STERNE, KESSLER, GOLDSTEIN & FOX, P.L.L.C., 1100 NEW YORK AVE., N.W., WASHINGTON, DC, 20005, US

(73) Assignee: Biogen Idec MA Inc., Cambridge, MA (US)

(21) Appl. No.: 11/666,221

(22) PCT Filed: Oct. 25, 2005
(86) PCT No.: PCT/US05/38714

§ 371 Date: Mar. 7, 2008

Related U.S. Application Data
(60) Provisional application No. 60/621,782, filed on Oct. 25, 2004.
(60) Provisional application No. 60/631,246, filed on Nov. 23, 2004.

(51) Int. Cl. A61K 39/00; C12N 15/00; C12N 5/00; A61K 48/00

(52) U.S. Cl. 424/233.1; 435/320.1; 435/325; 435/369; 435/456; 424/93.21

(57) ABSTRACT

This invention provides novel replication-defective adenoviral vectors comprising an adenoviral genome in which the protein IX gene, preferably under the control of its own promoter, is in an inverted orientation relative to the direction of transcription of the native protein IX gene at a location where the protein IX gene normally resides, for production of replication-competent adenovirus (RCA) free, or substantially RCA-free, adenovirus preparations. Said vector preferably encodes a gene of interest. The invention relates to viral particles, host cells and compositions comprising said adenoviral vector. This invention further relates a method for propagating adenovirus preparations, free, or substantially free, of replication-competent adenovirus (RCA) particles, from host cells comprising vectors of this invention, for use to treat a subject suffering from a disease or disorder or to prevent a subject from getting a disease or disorder, such as cancer. The invention also provides methods of treating such subjects and methods of prophylactically treating unaffected subjects. This invention further provides for vaccine compositions comprising the novel replication-defective adenoviral vectors of the present invention.

### TECHNICAL FIELD OF THE INVENTION

This invention relates to the field of gene therapy. Specifically, this invention relates to novel replication-defective adenoviral vectors comprising an adenoviral genome in which at least a part of the E1 region of the adenoviral genome is deleted and in which the protein IX gene is in an inverted orientation relative to the direction of transcription of the native protein IX gene at a location where the protein IX gene normally resides, wherein said recombinant adenoviral vector preferably comprises a gene of interest and said inverted protein IX gene and said gene of interest are operably linked to regulatory elements thereby allowing their expression in a host cell. This invention additionally concerns viral particles, host cells and compositions comprising said adenoviral vector. This invention further relates to a method and a system for propagating adenovirus preparations, free or substantially free of replication-competent adenovirus (RCA) particles, from the host cells of the invention, for use to treat a subject suffering from a disease or disorder or to prevent a subject from getting a disease or disorder, such as cancer. This invention further provides for vaccine compositions comprising the novel replication-defective adenoviral vectors of the present invention.

### BACKGROUND OF THE INVENTION

Gene therapy promises to treat or prevent a large number of diseases or disorders, such as cancer.

Viral vectors are used as delivery systems for gene therapy. A number of animal viruses are utilized as viral vectors. An adenoviral vector is a preferred viral vector for gene therapy. To be useful therapeutically, newly virally-derived gene therapy vectors require certain modifications to eliminate their disease-causing potential, yet retain their ability to replicate under controlled conditions to make viral preparations and to infect and deliver the desired therapeutic gene to the appropriate cell. Elimination of disease-causing potential of viral vectors may be achieved by deleting a subset of genetic elements from the viral genome to prevent independent viral replication in patients.

Adenoviral vectors are often constructed by insertion of a gene of interest in place of, or in the middle of, essential viral sequences such as those found at the E1 region (Berkner, BioTechniques, 6:616-629 (1988); Graham et al., Methods in Molecular Biology, 7:109-128, Ed: Murcy, The Human Press Inc. (1991)). Inactivation of essential viral genes by, for example, deletion or insertion, disables the adenovirus' ability to replicate. To propagate such vectors in cell culture, the deleted genes must be provided in trans (for example, the E1A and E1B proteins in the case of an E1 delete vector). These replication-defective adenoviruses are produced in packaging cells engineered to complement the replication-incompetent virus by expressing the subset of genetic elements deleted from their viral genome. Potential sites for the insertion of a gene of interest in recombinant adenoviral vectors include, without limitation, the E1, E2, E3 and the E4 region.

For example, a recombinant adenoviral vector may be derived from a human adenovirus and have the E1 region deleted and replaced with a heterologous gene of interest. The resulting viral vector, with one or more of its essential genes inactivated, will be replication defective (Statford-Perricaudet et al., Human Gene Therapy, 1:241-256 (1990)). Packaging cell lines such as the human embryonic kidney 293 (“HEK-293” or “293”) cell line (Graham et al., J. Gen. Virol., 36:59-72 (1977)) or human embryonic retinoblast (“HER-911” or “911”) cell line (Fallaux et al., Hum. Gene Ther., 7:215-222 (1996)), provide in trans the missing E1 region so that an E1-deleted or modified adenoviral vector can replicate in such cells.

The 293 cell line is the most commonly used packaging cell line for E1-minus adenoviral vectors. The 293 cells are derived from human embryonic kidney cells. The 293 cells comprise a portion of the adenovirus genome that includes the E1 region. Specifically, the 293 cells contain an approximately 4.3 kb region (1-4344) flanking the E1 locus of the adenoviral genome of the adenovirus 5 serotype. The 1-4344 nucleotide region of the 293 cells supports the replication of an E1-deleted replication-defective adenovirus very well, but these cells contain adenovirus sequences that extend beyond the E1 region.

A second E1 complementing cell line, 911, is derived from diploid human embryonic retinoblast (“HER”) cells and harbors nucleotides 80-5788 of the human adenovirus 5 genome (Fallaux et al., Hum. Gene Ther., 7:215-222 (1996)). The 911 cells exhibit matching qualities to that displayed by the 293 cells with respect to transfection efficiency and recombination frequency and are a useful alternative for the construction, propagation and titration of E1-deleted recombinant adenoviruses (Fallaux et al., Hum. Gene Ther., 7: 215-222 (1996)).

The 293 cell line has significant homology with the sequence of existing recombinant E1-minus adenoviral vectors on both sides of the gene-insertion cassette. These extended regions of nucleotide homology between the E1-minus adenoviral vector and the E1 region of the 293 cells could undergo homologous recombination, producing replication-competent adenovirus (“RCA”). The E1-positive contaminants (i.e., the RCA) eventually outgrow the original replication-deficient adenovirus in large-scale preparations (Lochmiller et al., Hum. Gene Ther., 5:1485-1491 (1994)). Hence, 293 cells comprising E1-deleted adenoviral vectors are not ideal for large scale production of clinical grade material, as batches frequently contain unacceptably high levels of RCA (Imler et al., Gene Ther., 3:75-84 (1994)). Even trace levels of RCA are a significant concern to regulatory agencies when these vectors are intended for human use. The emergence of RCA has been a major obstacle in large quantity vector production as well as having the potential of endangering the safety of gene therapy trials.

Several strategies have been developed to reduce or eliminate RCA contamination. One strategy involves the creation of new packaging cell lines that do not rely on the 293 cell line. The E1 portion contained in these cells have no homologous overlap with the E1 region of a specially designed adenoviral vector. A number of such cell lines were created (Fallaux et al., Hum Gene Ther., 9:1909-1917 (1998); Schiedner et al., Hum Gene Ther., 11: 2105-2116 (2000)). An example is the “Per.C6” cell line, disclosed in PCT publication WO 97/00326. Per.C6 consists of human embryonic retinoblasts (“HER”) cells stably transfected with a plasmid construct containing nucleotides 459-3510 of adenovirus 5 (“Ad5”). Prevention of RCA formation by the use of this cell line during the generation of E1-deleted adenoviral vectors requires the use of matching E1-deleted adenoviral vectors lacking Ad5 nucleotides 459-3510 (such vectors are also disclosed in WO 97/00326). But the problem is that existing E1-deleted adenoviral vectors undergoing clinical testing contain 3′ and 5′ boundaries at their E1 deletion that overlap with the adenoviral genomic portion of Per.C6 cells. Thus, to take advantage of this system, it would require reconstructing all of the adenoviral vectors currently being approved or seeking approval for use in human patients. Furthermore, due to still unclear reasons, helper dependent E1-positive adenoviral particles (“HDEP”) were detected during vector amplification in Per.C6 cells, regardless of the use of either matched E1-deleted adenoviral vectors or unmatched vectors (Murakami et al., Hum Gene Ther., 13: 909-920 (2002); Murakami et al., J Virol., 78: 6200-6208 (2004)).

PCT Publication WO 01/44280 provides an improvement to the Per.C6 system. This system provides stable complementing cell lines that encode and express adenoviral gene products without containing adenoviral genomes with overlapping homology to adenoviral vectors. Yet, the system does not require the use of a limited subset of matched vectors in order to avoid RCA formation. The cell lines disclosed in WO 01/44280 are recombinantly engineered to complement replication-incompetent adenoviruses because the cell lines are stably transformed or transfected with a complementation element comprising a nucleic acid molecule carrying, at a minimum, nucleotide sequences encoding one or more essential adenoviral proteins (such as, preferably, one or more proteins encoded by the E1 locus and most preferably, all of the proteins encoded by the Ad5 E1 locus including the Ad5 8.3 kDa E1b protein). WO 01/44280 further discloses that as a means of avoiding homology with the recombinant adenoviral vector and concomitantly reducing the likelihood of homologous recombination leading to RCA production, the complementation element comprises a non-naturally occurring adenovirus nucleotide sequence that still encodes an adenovirus protein. The problem with this system, however, is that it requires using the human diploid cells MRC-5 and WI-38 as complementary cell lines instead of the 293 cell line. As discussed above, the 293 cell line has already been developed and qualified for producing clinical material.

Another attempted strategy to reduce RCA production is to modify the viral vector backbone to reduce homology between the adenoviral vector and the E1 region of the 293 cells. Normally, the double crossovers between the E1 regions in 293 cells and the adenoviral vector occur in the protein IX and a partial IVa2 gene region. Two articles describe these adenoviral genome modifications. One involves protein IX gene relocation to the E3 region (Hehir et al., J. Virol., 70:8459-8467 (1996) (“Hehir”)); the other involves redesigning protein IX and IVa2 genes to reduce the homology by changing nucleotides conforming with codon degeneration to reduce the homology (i.e., making silent mutations) (Robert et al., Gene Ther., 8:1713-1720 (2001) (“Robert”)). Both articles reported some success in decreasing RCA production.

The gene encoding protein IX (“pIX”), a virion structural protein that is dispensable for growth and packaging of viral genomes that are less than 95% of wild-type length (Colby et al., J Virol., 39:977-980 (1981); Ghosh-Choudhury et al., EMBO J., 6:1733-1739 (1987)), comprises approximately 500 bp of this cross-over region between 293 adenoviral sequences and each of the adenoviral vector sequences. The adenoviral pIX is a minor component of the adenovirus capsid and is in part responsible for virion stability. Virions lacking pIX are heat labile and lose their infectivity if the DNA content is greater than 35 kb. pIX has been identified as a transcriptional activator and, in transient-transfection assays, was shown to enhance expression from the E1A, E4, and major late adenovirus promoters by as much as 70-fold (Colby et al., J Virol., 39:977-980 (1981); Ghosh-Choudhury et al., EMBO J., 6:1733-1739 (1987)). The major late promoter on the top strand of the virus and the IVa2 gene on the bottom strand of the virus are downstream of the pIX gene and must be retained for efficient vector growth.

As reported by Hehir, deletion of the pIX gene led to reduced RCA production. Hehir describes two approaches to modify the adenoviral vector backbone: either deleting or relocating the pIX coding region normally present downstream from the E1 region such that the recombination frequency between the vector and 293 Ad5 sequences is greatly reduced (Hehir et al., J Virol., 70:8459-8467 (1996). But deletion of the pIX sequences has two distinct disadvantages. First, the cloning capacity of such vectors is limited because the vector genome length must be less than 95% of the wild type adenoviral (adenovirus 2) genome length (Colby et al., J Virol., 39:977-980 (1981); Ghosh-Choudhury et al., EMBO J., 6:1733-1739 (1987)). For vectors carrying a large cDNA, at least a portion of the E3 (or another) region would also have to be deleted so as not to exceed the vector size limit. Second, the lack of pIX decreases the thermostability of the adenoviral vector (Colby et al., J Virol., 39:977-980 (1981); Ghosh-Choudhury et al., EMBO J., 6:1733-1739 (1987)). Therefore, Hehir explored an alternative strategy to reduce the recombination with 293 cell adenoviral sequences.

Hehir's second approach involved relocating the pIX sequence to a different part of the adenoviral vector. This strategy retains pIX gene and would allow the packaging of adenoviral vector genomes of full-length or greater size, which would in turn allow the retention of the E3 (or other) genes and large cDNAs, as well as obviating the lack of thermostability in a pIX-deleted vector. The relocation of the pIX gene reduced the occurrence of RCA in high titer vector preparations. Nevertheless, RCA was still observed (Hehir et al., J Virol., 70:8459-8467 (1996)). Also, this strategy calls for modifying existing adenoviral vectors.

Robert describes modified recombinant sequences introduced within the adenoviral vector backbone either by having point mutations in the pIX and IVa2 coding regions or modifying the noncoding pIX region (Robert et al., Gene Ther., 8:1713-1720 (2001)). Both approaches were able to decrease RCA emergence during amplification in 293 cells without altering vector productivity. But RCA contamination was still detectable.

Another approach to produce contaminant particle free adenoviruses was proposed in U.S. Pat. No. 6,312,946 (the '946 patent) and U.S. Pat. No. 6,482,617 (the '617 patent). The '946 and '617 patents provide for defective recombinant adenoviruses comprising an adenovirus genome whose genetic organization is modified such that any possible recombination with genome of the producing cell line leads to the generation of non-replicative and/or non-viable viral particles. The '946 and '617 patents achieves this by preparing recombinant adenoviruses comprising an E1 inactivated region whose genomic organization is modified such that genes or regions essential to viral replication and/or propagation is present in a genomic position other than its original position. Specifically, the genes or regions essential to viral replication and/or propagation, such as all or part of the E4 region, the pIX-IVa2 region, and the L5 region, are present at a genomic position other than its original position, for example, in the inactivated E1 and/or E3 regions. Thus, the replication defective recombinant adenoviruses of the '946 and '617 patents have an adenoviral genome comprising a first inactivated E1 region, a second inactivated region inactivated at its original location, chosen from the pIX-IVa2 region, the E4 region and the L5 region (or another region) and a functional region inserted at a position other than the original position which complements the second inactivated region containing all or part of an E4 region, a pIX-IVa2 region or an L5 region. However, there are many problems with this system. This system requires genomic reorganization of viral genes to be at genomic positions other than its original position. However, relocation of viral gene transcription, such as the pIX gene, to a distal site from its natural location in the viral genome may potentially alter the transcriptional control of the viral gene and may influence the properties of the vector. The system also involves extensive manipulation of the viral genome by initialing deleting and then subsequently relocating viral genomic regions, all of which requires modifying existing adenoviral vectors currently being approved or seeking approval for use in human patients.

Accordingly, there are still many problems with using 293 cells to produce substantially RCA-free recombinant adenoviral particles for gene therapy. The present invention provides a strategy for reducing RCA contamination in adenoviral preparations made from 293 cells comprising such vectors.

### SUMMARY OF THE INVENTION

This invention solves the problem outlined in the background section and others by providing a novel recombinant replication-defective adenoviral vector comprising an adenoviral genome in which at least a part of the E1 region of the adenoviral genome is deleted and in which the protein IX gene, operably linked to regulatory elements (thereby allowing its expression in a host cell), is in an inverted orientation relative to the direction of transcription of the native protein IX gene at a location where the protein IX gene normally resides. In certain embodiments, such a recombinant adenoviral vector also comprises a gene of interest such that the inverted protein IX gene and the gene of interest are operably linked to one or more regulatory elements thereby allowing their expression in a host cell. The adenoviral vector may have all of the E1 region of the adenoviral genome deleted. The adenoviral vector may have the inverted protein IX gene placed under the control of the protein IX natural promoter. The adenoviral vector may have the gene of interest placed under the control of the cytomegalovirus immediate early promoter (CMV promoter). The adenoviral vector may also have the gene of interest under the control of other promoters including the dihydrofolate reductase promoter, the pyruvate kinase promoter, the β-actin promoter, the early and late promoters of SV40, the long terminal repeats of Moloney Leukemia Virus and other retroviruses, the thymidine kinase promoter of the Herpes Simplex Virus (HSV), the CMV immediate-early (IE1) promoter, the promoter of the Rous sarcoma virus (RSV), the adenovirus major late promoter, the liver-specific promoters of hepatitis-β virus, the mammary carcinoma specific β-casein promoter, the melanoma-specific tyrosinase promoter, the osteosarcoma-specific c-sis promoter, and the glioma and neuroblastoma-specific calcineurin A alpha promoter. The adenoviral vector may have the gene of interest encode a protein or fragment or portion thereof having substantial identity with said protein, usually at least about 70% identity, preferably at least about 75% identity, more preferably about 80% identity, and most preferably at least about 95% identity, selected from the group consisting of interferon-β, herpes simplex virus thymidine kinase and p53, preferably interferon-β, and more preferably human interferon-β.

This invention also provides a recombinant replication-defective adenoviral vector comprising an adenoviral genome which is wild-type except for a deletion in at least a part of the E1 region and in at least a part of the E3 region of the adenoviral genome and in which the adenoviral genome has the protein IX gene in an inverted orientation relative to the direction of transcription of the native protein IX gene at a location where the protein IX gene normally resides, wherein said recombinant adenoviral vector preferably comprises a gene of interest; wherein said inverted protein IX gene and said gene of interest are operably linked to regulatory elements thereby allowing their expression in a host cell. This adenoviral vector may have all of the E1 region of the adenoviral genome deleted. This adenoviral vector may have the E3 region deleted from adenovirus 5 map unit 83.3 through map unit 85.4 (or nucleotide 30005-30750, Bett et al., Virus Res., 39:75-82 (1995)). This adenoviral vector may have the inverted protein IX gene placed under the control of the protein IX natural promoter. In certain embodiments, this adenoviral vector further comprises a deletion of all or a part of the IVa2 gene, replacement of a part or all of the IVa2 gene with another domain (as described herein) or an inversion of the IVa2 gene at the location where the IVa2 gene normally resides. This adenoviral vector may have the gene of interest placed under the control of the cytomegalovirus immediate early promoter (CMV promoter). This adenoviral vector may also have the gene of interest under the control of other promoters including the dihydrofolate reductase promoter, the pyruvate kinase promoter, the β-actin promoter, the early and late promoters of SV40, the long terminal repeats of Moloney Leukemia Virus and other retroviruses, the thymidine kinase promoter of the Herpes Simplex Virus (HSV), the CMV immediate-early (IE1) promoter, the promoter of the Rous sarcoma virus (RSV), the adenovirus major late promoter, the liver-specific promoters of hepatitis-B virus, the mammary carcinoma specific β-casein promoter, the melanoma-specific tyrosinase promoter, the osteosarcoma-specific c-sis promoter, and the glioma and neuroblastoma-specific calcineurin A alpha promoter. The adenoviral vector may have the gene of interest encode a protein or fragment or portion thereof having substantial identity with said protein, usually at least about 70% identity, preferably at least about 75% identity, more preferably about 80% identity, and most preferably at least about 95% identity, selected from the group consisting of interferon-β, herpes simplex virus thymidine kinase and p53, preferably interferon-β, and more preferably human interferon-P. This adenoviral vector may have the gene of interest encode a protein or fragment or portion thereof selected from the group consisting of interferon-β, herpes simplex virus thymidine kinase and p53, preferably interferon-β, and more preferably human interferon-β. This adenoviral vector may be AdIFNβ-RIX.

This invention also provides a recombinant replication-defective adenoviral vector comprising an adenoviral genome which is wild-type except for a deletion in at least a part of the E1 region and in at least a part of the E3 region of the adenoviral genome in which the E1 region is deleted between nucleotide base pairs 353 and 3332 of the adenoviral genome and in which the adenoviral genome has the protein IX gene in an inverted orientation relative to the direction of transcription of the native protein IX gene at a location where the protein IX gene normally resides. This adenoviral vector may have the inverted protein IX gene placed under the control of the protein IX natural promoter. In certain embodiments, this adenoviral vector further comprises a deletion of all or a part of the IVa2 gene, replacement of a part or all of the IVa2 gene with another domain (as described herein) or an inversion of the IVa2 gene at the location where the IVa2 gene normally resides. This adenoviral vector may further comprise a gene of interest that is inserted into the adenoviral genome at a location where the E1 region is deleted between nucleotide base pairs 353 and 3332 of the adenoviral genome. This adenoviral vector may have the gene of interest and the inverted protein IX gene operably linked to regulatory elements thereby allowing their expression in a host cell. This adenoviral vector may have the gene of interest placed under the control of the cytomegalovirus immediate early promoter (CMV promoter). This adenoviral vector may also have the gene of interest under the control of other promoters including the dihydrofolate reductase promoter, the pyruvate kinase promoter, the β-actin promoter, the early and late promoters of SV40, the long terminal repeats of Moloney Leukemia Virus and other retroviruses, the thymidine kinase promoter of the Herpes Simplex Virus (HSV), the CMV immediate-early (IE1) promoter, the promoter of the Rous sarcoma virus (RSV), the adenovirus major late promoter, the liver-specific promoters of hepatitis-β virus, the mammary carcinoma specific β-casein promoter, the melanoma-specific tyrosinase promoter, the osteosarcoma-specific c-sis promoter, and the glioma and neuroblastoma-specific calcineurin A alpha promoter. This adenoviral vector may have the gene of interest encode a protein or fragment or portion thereof having substantial identity with said protein, usually at least about 70% identity, preferably at least about 75% identity, more preferably about 80% identity, and most preferably at least about 95% identity, selected from the group consisting of interferon-β, herpes simplex virus thymidine kinase and p53, preferably interferon-β, and more preferably human interferon-β. This adenoviral vector may have the gene of interest encode a protein or fragment or portion thereof selected from the group consisting of interferon-β, herpes simplex virus thymidine kinase and p53, preferably interferon-β, and more preferably human interferon-β. This adenoviral vector may be AdIFNβ-RIX.

The invention also provides a viral particle comprising the recombinant adenoviral vector of this invention.

The invention also provides an isolated host cell comprising the adenoviral vector of this invention. The host cell may be a mammalian cell and in certain embodiments, that mammalian cell may be a human cell. The host cell may be a human cell that is a human embryonic kidney 293 cell. The host cell's genome may have insufficient overlapping sequences between it and an adenoviral vector genome to appreciably mediate a recombination event sufficient to result in a replication-competent adenovirus, wherein the overlapping sequences between said adenoviral genome and said host cell genome is no more than 633 bp, preferably no more than 617 bp. More preferably, the right-arm region homologous sequences between said adenoviral vector genome and said host cell genome is no more than 280 bp, preferably no more than 264 bp.

This invention also relates to a preparation that is substantially free of replication-competent adenovirus comprising the adenoviral vector of this invention, wherein said adenoviral vector is prepared in a human embryonic kidney 293 cell which supports the growth of said adenoviral vector.

The invention also relates to a method of propagating a replication-defective adenoviral particle of this invention, comprising introducing the adenoviral vector of this invention into a host cell, culturing said host cell comprising said vector for an appropriate period of time and under suitable conditions to allow the production of said viral particle, recovering said viral particle from said culture, and optionally, purifying said recovered viral particle. The host cell of this method may be a mammalian cell, which may be a human cell. That human cell may be a human embryonic kidney 293 cell. In certain embodiments, an adenoviral vector of the method may have insufficient overlapping sequences between said adenoviral vector genome and said host cellular genome to appreciably mediate a recombination event sufficient to result in a replication-competent adenovirus. In certain embodiments, the overlapping right-arm region homologous sequences between said adenoviral vector genome and said host cell genome is no more than 280 bp, preferably no more than 264 bp.

The invention also relates to a system comprising a host cell which complements in trans a deficiency in one or more essential gene functions of the E1 region of an adenoviral genome of an adenoviral vector of this invention, wherein there is insufficient overlapping sequences between said host cell genome and said adenoviral vector genome to appreciably mediate a recombination event sufficient to result in a replication-competent adenovirus. The system may comprise a host cell that is a human embryonic kidney 293 cell. In certain embodiments, the overlapping sequences located in the right-arm region between said host cell genome and said adenoviral vector genome that is no more than 280 bp, preferably no more than 264 bp.

The invention also provides a pharmaceutical composition comprising the adenoviral vector of this invention, and a pharmaceutically acceptable carrier.

The invention also relates to a method for treating cancer by in vivo gene therapy comprising the steps of administering to a subject an adenoviral vector of this invention or the viral particle of this invention comprising an adenoviral vector of this invention, and allowing said vector to express a gene of interest in said subject, in an amount sufficient to cause cancer regression or inhibition of cancer growth in part or in full, either alone or in combination with another agent. In certain embodiments, said gene of interest encodes interferon-β (preferably human interferon-β), herpes simplex virus thymidine kinase or p53 or fragments or portions thereof. In certain embodiments, the adenoviral vector or viral particle is administered by topical administration, intraocular administration, parenteral administration, intranasal administration, intratracheal administration, intrabronchial administration or subcutaneous administration. In certain embodiments, the adenoviral vector or viral particle is administered by direct injection at or near a site of a tumor in said subject. In certain embodiments, the cancer to be treated is malignant glioma, melanoma, hemanglioma, leukemia, lymphoma, myeloma, colorectal cancer, non-small cell carcinoma, breast cancer or ovarian cancer. In certain embodiments, the subject is human.

The invention also provides a vaccine composition comprising the adenoviral vector of this invention.

### BRIEF DESCRIPTION OF THE DRAWINGS

FIG. 1 is a schematic representation of the generation of recombination-competent adenovirus (“RCA”) via homologous recombination between an exemplary adenoviral vector (i.e., an E1 delete vector encoding human interferon-β (hIFNβ) under the control of the CMV promoter) and the 293 cell genome. One such adenoviral vector is AdIFNβ. The double crossovers occur between an approximately 353 nucleotide overlap region on the left-arm region of the Adenovirus 5 (“Ad5”) sequence and an approximately 1012 nucleotide overlap region spanning the pIX gene and a partial IVa2 gene on the right-arm region of the Ad5 sequence. FIG. 1 depicts such an exemplary adenoviral vector which comprises, in the order from the left-arm region of the adenoviral genome, the inverted terminal repeat (“ITR”), the virus packaging signal and enhancer sequences (“ψ”), the cytomegalovirus immediate early 1 gene enhancer and promoter (“CMV”), the SV40 intron sequence (“ivs”), the human interferon-beta gene (“hIFNβ”), the SV40 polyadenylation signal (“poly A”), the whole promoter/E1B enhancer region (“E1B”), the pIX gene (“pIX”), the protein Iva2 gene (“pIVa2”) and the reminder of the adenoviral genome sequences.

FIG. 2 is a schematic representation of overlap design and primer location to create the inverted pIX (“RIX”) gene that is fused to the 3′ end of the IVa2 gene. The inversion of nucleotides 3333-4031 in the parental vector was accomplished by an overlapping PCR strategy to redesign the sequences between the unique SalI and BstXI sites. A unique HindIII (“H3”) site between the 3′ end of the IVa2 gene and the inverted pIX gene sequence was created for the insertion of a polyadenylation signal to terminate the IVa2 gene transcription. FIG. 2A shows generation of an E1bRIX-H3-IVa2 fragment (which contains the EIB/pIX promoter) using the following primers: E1BRIX-H3-IVa2 top, E1BRIX-H3-IVa2 bot, IVa2 bot and RIX bot. FIG. 2B shows generation of a RIX-H3-IVa2 fragment (which contains the pIX promoter only) using the following primers: RIX-H3-IVa2 top, RIX-H3-IVa2 bot, IVa2 bot and RIX bot.

FIG. 3 is a schematic representation of the pIX vector design and viral genome structure using two different strategies. The adenoviral vector includes (in order from the left end of the adenoviral genome): the 5′ Inverted Terminal Repeats (“ITR”), the virus packaging signal and enhancer sequences (“ψ”), the CMV enhancer/promoter (“CMV”), the SV40 intron sequence (“ivs”), the human IFNβ gene (“hIFNβ”), the SV40 polyadenylation site (“polyA”), the inverted pIX gene (“pIX”) and either an internal ribosomal entry sequence (“IRES”) followed by the protein IVa2 gene (“pIVa2”), as shown in FIG. 3A, or a pIX promoter followed by the human growth hormone polyadenylation signal (“pA”) and the pIVa2 gene, as shown in FIG. 3B. FIG. 3A shows the inversion of the pIX gene using the pIX IRES approach. FIG. 3B shows the inversion of the pIX gene along with its natural promoter. The amount of overlap between the 293 cellular genome's adenoviral sequence and the adenoviral vector genome is also shown (i.e., 353 nucleotides on the left-arm region and 280 nucleotides and 264 nucleotides, respectively, on the right-arm region for the approaches shown in FIGS. 3A and 3B).

FIG. 4 is a graphical representation of four clones from four inverted pIX gene (“AdIFNβ-RIX”) plaque isolates. The isolates (4a to 4d; 20a to 20d; 22a to 22d and 31a to 31d) were used to infect A549 human lung carcinoma cells and the expression of human IFNβ transgene was determined by ELISA as described below. All four clones express human IFNβ at levels that were comparable to that of the parental vector. Positive controls containing dilutions of the protein were run in the presence of untransduced cell supernatants to control for media effects on detection level.

FIG. 5 is a graphical representation of IFNβ expressions levels from parental human IFNβ vector AdIFNβ and the inverted pIX (AdIFNβ-RIX) vector as a function of the multiplicity of infection (MOI). No significant difference was detected between the AdIFNβ-RIX vector and the parental AdIFNβ vector.

FIG. 6 is a graphical representation of the real time PCR (Q-PCR) results of E1 detection in serial passages of viral stocks. Parental human IFNβ vector (AdIFNβ), a control vector (AdIFNβ-M9, which is derived from the parental human IFNβ vector containing mutations in the pIX gene) and three inverted pIX (AdIFNβ-RIX) vectors, were serially passed three times (Master Seed Stock (MSS)—MSS1, MSS2 and MSS3) in 293 cells. The viral DNA was isolated and E1 gene copies were determined using Q-PCR. No E1 sequence was detected in all three AdIFNβ-RIX stocks.

FIG. 7 is a graphical representation of the real time PCR (Q-PCR) results of E1 detection in serial passages of viral stocks. Parental human IFNβ vector (AdIFNβ) and the AdIFNβ-RIX vectors were serially passed up to seven passages in 293 cells. RCA emergence was delayed by three passages in AdIFNβ-RIX as compared to AdIFNβ, which is estimated to be a 10,000 fold reduction in RCA incidence.

FIG. 8 is a table summarizing the liquid culture results. The same lots of viruses used in the Q-PCR assay described in FIG. 6 were analyzed by liquid culture method for the presence of RCA. “+” symbols indicate the levels of RCA detected. Parental vector (AdIFNβ 1-3), inverted pIX vector (AdIFNβ-RIX 1-3) and control vector (AdIFNβ-M91-3, which is a clone of the AdIFNβ-M9 vectors described in FIG. 6 above) were serially passaged in 293 cells up to passage 11 (P11) and then subjected first to absorption on HeLa cells. Next, any putative RCA would be transferred to A549 cells followed by culturing to amplify replicative recombinants. Finally, culture supernatants were assayed on naive A549 cells to verify presence of replicative virions and the results are displayed in the table as “Infection Test (supernatants from day 16 on fresh A549)”. No RCA was detected in all three AdIFNβ-RIX stocks by this method and these results confirmed the above Q-PCR data. This is indicated in the last column by the lack of cytopathic effect (“CPE”).

FIG. 9 are photomicrographs from the liquid culture assay displaying the cell morphology at day 7 after infection of HeLa cells with the wild type adenovirus 5 (“Ad5”) in the presence of AdIFNβ vector (to control for the IFNβ effects on plaquing efficiency). The top panels display the cell morphology of negative controls in which either media control or 3 mL of crude viral lysates (“CVL”) containing the adenoviral vector of this invention was added. The positive controls, shown in the bottom panels, display the morphology of cells containing early passage CVL (with no evidence of RCA) and spiked with either 5 plaque forming units (“pfu”), 50 pfu or 500 pfu of the wildtype Ad5. As low as 5 pfu can be detected in this assay in the presence of IFNβ expression.

FIG. 10 shows the liquid culture results of inverted pIX (AdIFNβ-RIX) vectors and parental human IFNβ vector (AdIFNβ)). The photomicrographs in the top panel pictures show the A549 cell infection results, indicating the absence of RCA in AdIFNβ-RIX stocks (MSS3). The photomicrographs in the lower panel show the confirmation result of infection of fresh naive A549 cells using the lysate from the original A549 test. This further confirms the absence of RCA in AdIFNβ-RIX containing cells and ruled out the possibility of IFNβ induced cell death effects.

FIG. 11 is a table summarizing the liquid culture results of inverted pIX (AdIFNβ-RIX) vectors and parental human IFNβ vector (AdIFNβ)). The emergence of RCA was delayed three passages in AdIFNβ-RIX vector as compared to AdIFNβ.

FIG. 12 is a table summarizing the liquid culture results from small scale culture passage experiments of inverted pIX (AdIFNβ-RIX) vectors and parental human IFNβ vector (AdIFNβ)). Reduced incidence of RCA was observed for the AdIFNβ-RIX vector with RCA emerging at passage 21 whereas cells infected with the AdIFNβ vector displayed RCA as early as passage 19.

FIG. 13 is a graphical representation of the thermostability of inverted pIX (AdIFNβ-RIX) vectors and parental human IFNβ vector (AdIFNβ) over time. There was no significant difference between the AdIFNβ-RIX and AdIFNβ vectors at any incubation time.

FIG. 14 is a graphical representation of the thermostability of inverted pIX (AdIFNβ-RIX) vectors and parental human IFNβ vector (AdIFNβ) with increasing incubation temperatures. There was no statistical difference between the AdIFNβ-RIX and AdIFNβ vectors with increasing incubation temperatures.

FIG. 15 displays results of the viral particle analysis by HPLC. The panels include viral particles containing the parental human IFNβ vector (AdIFNβ) and an inverted pIX gene vector (AdIFNβ-RIX). The results indicate that the viral particles are indistinguishable from the parental vector (AdIFNβ).

FIG. 16 displays results of the viral particle analysis by analytical ultra-centrifugation (AUC). The panels include viral particles containing the parental human IFNβ vector (AdIFNβ) and an inverted pIX gene vector (AdIFNβ-RIX). The results indicate that the AdIFNβ-RIX viral particles are indistinguishable from the parental vector(AdIFNβ).

FIG. 17 shows the results of testing the inverted pIX (AdIFNβ-RIX) vector in a xenograft tumor model in nude mice for tumor killing activity. Briefly, U87 cells, a human glioma cell line, were injected subcutaneously into nude mice to establish tumor. One week after cell implantation, tumors were treated with the AdIFNβ-RIX vector (1×10¹¹ viral particles/tumor), parental human IFNβ vector (AdIFNβ) (1×10¹¹ viral particles/tumor) and control vector (AdLacZ) (1×10¹¹ viral particles/tumor). Both the AdIFNβ-RIX and parental vectors showed comparable tumor killing activities, indicating that modification of the pIX gene did not alter either the IFNβ expression levels or the AdIFNβ tumor killing capability.

FIG. 18 displays the nucleotide sequence of the recombinant adenoviral vector of this invention containing the inverted pIX gene, AdIFNβ-RIX. AdIFNβ-RIX is also named H5R9CMVIFNβ (SEQ ID NO: 1) and is 35865 base pairs in length. The recombinant adenovirus produced from this vector, and comprising this vector, is deposited with the American Type Culture Collection (“ATCC”) on Sep. 10, 2004, and has ATCC Accession No. PTA-6198.

FIG. 19 displays an additional nucleotide sequence encoding the AdIFNβ-RIX recombinant adenoviral vector of this invention (SEQ ID NO: 23). This sequence is a shorter version of SEQ ID NO: 1 and is 35849 base pairs in length.

### DETAILED DESCRIPTION OF THE INVENTION

Unless otherwise required by context, singular terms shall include pluralities and plural terms shall include the singular. Throughout this specification and embodiments, the word “comprise,” or variations such as “comprises” or “comprising,” will be understood to imply the inclusion of a stated integer or group of integers but not the exclusion of any other integer or group of integers.

The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), the disclosures of all of which are incorporated herein by reference. Standard techniques are used for pharmaceutical preparation, formulation, and delivery, and treatment of patients.

For techniques related to adenovirus, see, for example, Felgner and Ringold (1989) Nature 337: 387-388; Berkner and Sharp (1983) Nucl. Acid Res. 11:6003-6020; Graham (1984) EMBO J. 3:2917-2922; Bett et al. (1994) Proc. Natl. Acad. Sci. USA 91:8802-8806, the disclosures of all of which are incorporated herein by reference.

### Adenoviral Vectors

Adenovirus is a suitable vector for gene therapy. See, e.g., PCT publication WO 99/10516, published Mar. 4, 1999, the disclosure of which is hereby incorporated by reference. The adenovirus-based vector could be from any adenovirus derived from any organism (such as human) and from any serotype, such as, the human adenovirus serotype 5 (“Ad5”) or serotype 2 (“Ad2”). See, e.g., Muzyczka (1992) Curr. Top. Microbiol. Immunol. 158: 97-123; Ali et al. (1994) Gene Therapy 1:367-384; U.S. Pat. Nos. 4,797,368 and 5,339,346.

Adenoviruses could be modified to efficiently deliver a therapeutic (which includes a gene delivered for prophylactic purposes) or reporter transgene to a variety of cell types. For example, the general adenoviruses types 2 and 5 (Ad2 and Ad5, respectively), which cause respiratory disease in humans, are currently being developed for clinical trials, including treatment of cancer or other cell proliferation diseases and disorders, and for gene therapy of Duchenne Muscular Dystrophy (DMD) and Cystic Fibrosis (CF). Both Ad2 and Ad5 belong to a subclass of adenovirus that are not associated with human malignancies. Adenovirus vectors are capable of providing high levels of transgene (also referred to herein as gene of interest) delivery to diverse cell types, regardless of the mitotic state of the cell. High titers (1013 plaque forming units/ml) of recombinant virus can be easily generated in 293 host cells (an adenovirus-transformed, E1 complementation human embryonic kidney cell line: ATCC No. CRL1573) and cryo-stored for extended periods without appreciable losses. The efficacy of this system in delivering a therapeutic transgene in vivo that complements a genetic imbalance has been demonstrated in animal models of various disorders. See, e.g., Watanabe, 1986 Atherosclerosis, 36: 261-268; Tanzawa et al., 1980 FEBS Letters, 118(1): 81-84; Golasten et al., 1983 New Engl. J. Med., 309: 288-296; Ishibashi et al., 1993 J. Clin. Invest., 92: 883-893; Ishibashi et al., 1994 J. Clin. Invest., 93: 1889-1893, the disclosures of all of which are incorporated herein by reference. Recombinant replication-defective adenovirus encoding a cDNA for the cystic fibrosis transmembrane regulator (CFTR) gene product has been approved for use in at least two human Cystic Fibrosis clinical trials. See, e.g., Wilson, 1993 Nature, 365: 691-692.

The strong immunogenicity and high levels of transient gene expression of recombinant adenoviral vectors makes them attractive for recombinant vaccines (Brandsma et al. (2004) J. Virol. 78:116-123; Edelman et al. (2003) Curr. Opin. Mol. Ther. 5:611-617; Gomez-Roman et al. (2003) AIDS Rev. 5:178-185; Odaka et al. (2001) Cancer Res. 61:6201-6212; St George, J. A. (2003) Gene Ther. 10:1135-1141; Tada et al. (2001) J. Clin Invest. 108:83-95; Qin et al. (1998) Proc Natl Acad Sci USA. 95(24):14411-14416; Nadeau et al. (2003) Biotechnol Adv. 20(7-8):475-489). A number of recombinant adenoviral vector based cancer gene therapy trials have been approved in the U.S., of which two use adenoviral vectors to express human interferon-β (IFNβ).

Some replication-deficient adenoviral vectors developed for clinical trials contain deletions of the entire E1a and part of the E1b regions. These vectors are typically grown in 293 cells, which contain functional adenoviral E1a and E1b genes, thus providing to the vectors the E1a and E1b proteins in trans. These replication-defective adenoviral vectors can thus grow into adenovirus in 293 cells (Graham et al. (1977) J Gen Virol. 36(1):59-74). The resulting adenovirus comprising the adenoviral vector is capable of infecting many cell types and can express the gene of interest carried on the adenoviral vector, provided that the gene of interest carries appropriate expression control elements. But the replication-defective virus cannot replicate in a cell that does not carry the adenoviral E1 region DNA.

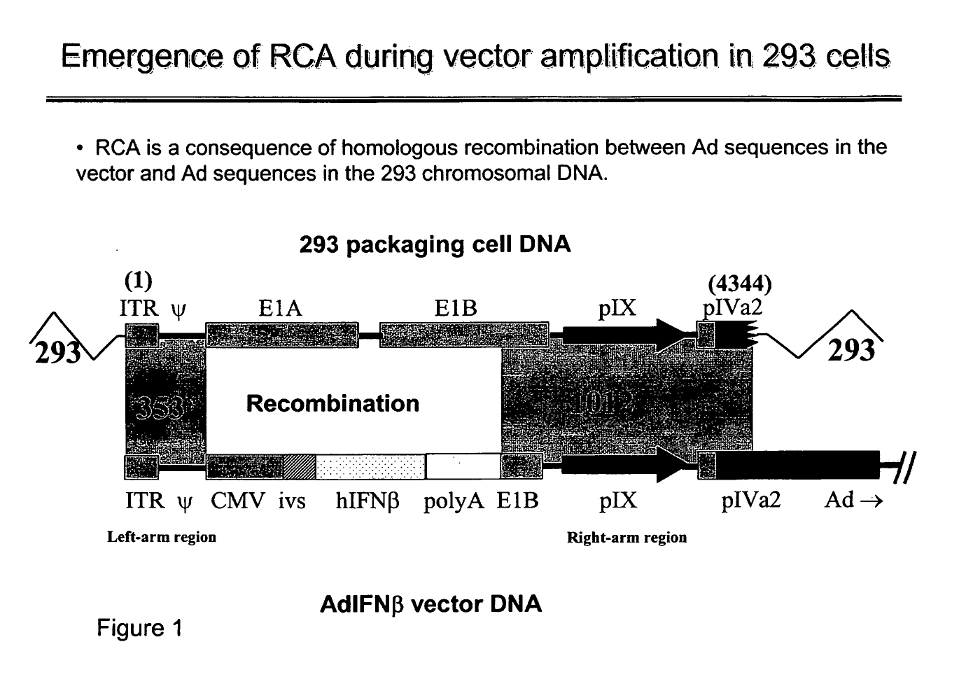

The emergence, however, of replication-competent adenoviruses (“RCA”) in high titer recombinant adenoviral stock raises safety concerns and has been a major concern in large-scale vector production (Fallaux et al. (1999) Gene Ther. 6:709-712). RCA is generated during adenoviral vector propagation in 293 host cells from the original adenoviral vector. RCA reacquires the E1 sequences from the 293 host cells via homologous recombination between the adenoviral vector and the 293 cell genome (Hehir et al. (1996) J. Virol. 70:8459-8467; Louis et al. (1997) Virology 233:423-429; Zhu et al. (1999) Gene Ther. 10:113-121) and becomes E1-independent and replication-competent in non-E1 expressing cells. The double crossovers occur between the E1 region (Ad5 nucleotides 1-4344) in 293 cells and its corresponding adenovirus sequence in the adenoviral vector. Specifically, the adenoviral vector sequences involved include the Ad5 left-arm region of approximately 350 bp and the Ad5 right-arm region of approximately 1 kb which spans a region that extends over the entire protein IX gene and a partial IVa2 gene. See FIG. 1. The exact length of these two regions may vary in different adenoviral vector constructs. As a result of homologous recombination, the transgene expression cassette in the adenoviral vector is replaced by the adenoviral E1 sequence and thus, is devoid of the transgene. The E1 containing virus has growth advantages over the recombinant adenoviral vectors, leading to RCA populating itself quickly and becoming dominant in adenoviral vector stocks after its emergence. RCA contamination is clearly undesirable, and the NIH and FDA have developed strict guidelines to monitor and control the RCA contamination levels in adenoviral vector materials intended for clinical use.

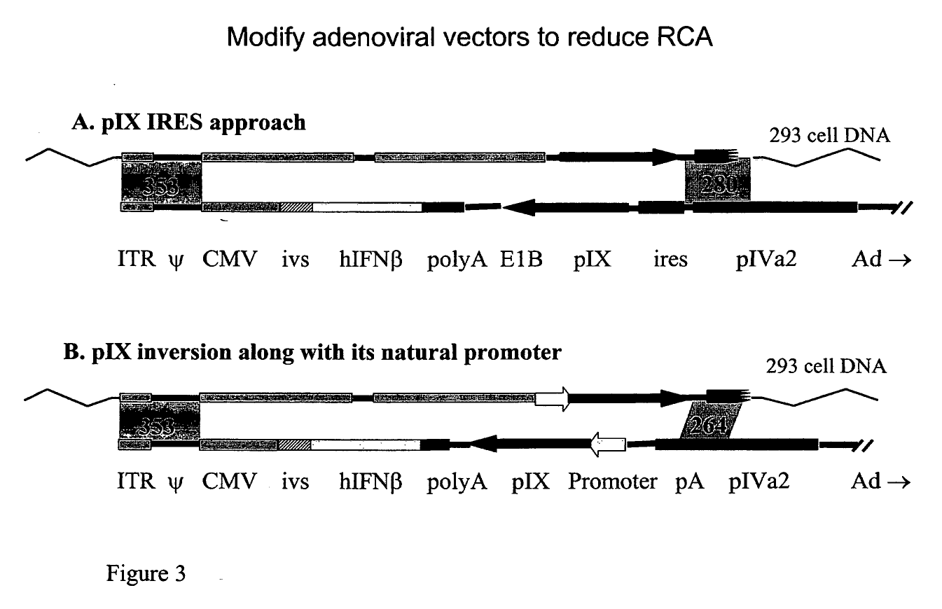

To reduce RCA production, this invention provides a novel strategy to reduce the homology between the adenoviral backbone and the E1 sequence in 293 cellular genome, allowing the use of 293 cells and without resorting to the expense of large-scale alteration of the pIX gene or the undesirability of moving the pIX gene to another location. Specifically, to break the continuity of all or some portion of the homologous sequences responsible for RCA generation, the pIX gene is inverted at its natural location. The inversion of the pIX gene in the design of the adenoviral vector construct significantly reduces the overlapping region with 293 host cell E1 sequences at the pIX-IVa2 gene region. See FIG. 3.

This invention provides a recombinant adenoviral vector comprising an adenoviral genome in which the pIX gene is in an inverted orientation relative to the direction of transcription of the native pIX gene at a location where the pIX gene normally resides. In certain embodiments, the inverted pIX gene of this invention is placed under the control of its natural pIX promoter.

As shown in Example 1, such an adenoviral vector comprises an inverted pIX gene at its natural location from nucleotides 3333-4031 in the adenoviral vector. Example 1 describes two approaches undertaken to invert the pIX gene in the parental adenoviral vector (an adenoviral vector bearing deletions in E1 and E3 regions and encoding human interferon-β referred to herein as AdIFNβ (Qin et al., Proc Natl Acad Sci USA., 95(24):14411-14416 (1998)), of which only the second approach resulted in successful creation of the AdIFNβ-RIX (this represents the AdIFNβ vector with an inverted pIX gene). As used herein, the term “AdIFNβ” is also known as BG00001. These terms refer to the same adenoviral vector. Similarly, as used herein, the term “AdIFNβ-RIX” is also known as R9 or RIX. These terms refer to the same adenoviral vector. Specifically, the first pIX gene inversion strategy involves the use of an internal ribosomal entry sequence (“IRES”) which allows tethering the pIX expression to that of the pIVa2 gene. Using this first approach, the adenoviral vector contains the pIX gene in an inverted orientation at its natural location with its expression being directed by the IVa2 gene promoter and an IRES sequence. This first inversion approach would result in a significant reduction of the overlapping region with 293 host cell E1 sequences at the pIX-IVa2 gene region (i.e., right-arm region) such that homology at the Ad5 left-arm region would still be approximately 353 bp but the homology at the Ad5 right-arm region would be reduced from approximately 1 kb to approximately 280 bp. Thus, the total overlapping sequences between the adenoviral genome and the 293 host cellular genome would be 633 bp. However, this inversion technique did not result in the generation of recombinant replication-defective adenoviruses.

Example 1 also describes the second strategy to generate recombinant replication-defective adenoviruses by utilizing a direct inversion approach of the pIX gene transcription unit in its natural location and having the pIX gene expression be directed by its own pIX natural promoter. This second inversion strategy was accomplished by an overlapping PCR strategy to redesign the sequence between the unique SalI and Bst XI sites and resulted in an adenoviral vector comprising (in order from the left-arm region of the adenoviral genome) the inverted terminal repeats (“ITR”), the virus packaging signal and enhancer signals (“ψ”), the CMV promoter (“CMV”), the human IFNβ gene (hIFNβ), the bi-directional SV40 polyA (“polyA”), and the pIX gene (inverted), pIX gene minimal promoter or the whole promoter/E1B enhancer region, the human growth hormone polyadenylation site (“pA”), and the protein IVa2 (“pIVa2”) gene. This second inversion approach would result in a significant reduction of the overlapping region with 293 host cell E1 sequences at the pIX-IVa2 gene region such that homology at the Ad5 left-arm region would still be approximately 353 bp but the homology at the Ad5 right-arm region would be reduced from approximately 1 kb to approximately 264 bp. Thus, the total overlapping sequences between the adenoviral genome and the 293 host cellular genome would be 617 bp. This latter approach resulted in the generation of recombinant replication-defective adenoviruses without the emergence of RCA.

In certain embodiments, a recombinant adenoviral vector is a vector that is rendered replication-defective by deletion or modification of one or more essential genes of the adenovirus (such as the deletion of one or more adenoviral E1 genes). In certain embodiments, the adenoviral vector comprises an adenoviral genome in which at least a part of the E1 region of the adenoviral genome is deleted. In certain embodiments, all of the E1 region of the adenoviral genome is deleted. This recombinant adenoviral vector produces recombinant replication-defective adenovirus particles in the host cells of this invention. The adenovirus particles so produced are used, for example, for gene therapy in vivo, as detailed below.

In certain embodiments, a recombinant adenoviral vector comprises a heterologous gene of interest, which is a non-adenoviral gene. This heterologous gene encodes a gene product, which may be a protein, or an RNA. The therapeutic or diagnostic use intended for the resulting adenoviral preparation or composition dictates the nature of the heterologous gene of interest encoded by the adenoviral vector.

A recombinant adenoviral vector that comprises a heterologous gene of interest can be an expression vector. That vector allows expression of the heterologous genes of interest in one or more cells of a subject.

In certain embodiments, the adenoviral vector is the parent human IFNβ adenoviral vector (referred to herein as AdIFNβ) with an inverted pIX gene at its natural location (referred to herein as AdIFNβ-RIX). The map of AdIFNβ is shown in FIG. 1 (see, AdIFNβ vector DNA). The nucleotide base numbers used in the description of the adenoviral sequences refer to the Adenovirus 5 genome sequence listed in Genbank under accession number M73260. AdIFNβ is an adenoviral vector that comprises all of the human adenovirus 5 genome, except for a deletion in the E1 region from nucleic acid residue 359 to 2954 and a small deletion in the E3 region between nucleic acid residues 30005 and 30750. AdIFNβ bears a human interferon-β gene (IFNβ) under the control of the cytomegalovirus immediate-early (CMV-IE) promoter/enhancer. As shown in FIG. 1, this IFNβ gene is inserted in the E1 region, replacing the E1 region that is deleted. The adenoviral vector of this invention (such as AdIFNβ-RIX) further comprises an inverted pIX (“pIX”) gene at its natural location, wherein the pIX gene is inverted from nucleic acid residues 3333-4031 in the parental adenoviral vector. In certain embodiments of this invention, the inverted pIX (“pIX”) gene of this invention is placed under the control of its natural pIX promoter (such as AdIFNβ-RIX).

Accordingly, the reduced incidence of RCA observed with the AdIFNβ-RIX vector has provided scientific support to the use of the “gene inversion strategy” as an effective means to improve current adenoviral vectors. However, there still remains two small overlapping regions between the AdIFNβ-RIX vector backbone and the 293 cell genome which may potentially contribute to the emergence of RCAs. One of the overlapping regions is the 353 bp on the left end of the viral genome and the other overlapping region is the 264 bp sequence following the inverted pIX gene as shown in FIG. 3.

Because the two overlapping regions may contribute to the eventual emergence of RCA, additional manipulation of the 264 bp region to decrease its homology with the 293 cells will further reduce or even completely eliminate the RCA.

One approach involves inverting the protein IVa2 gene. The product of protein IVa2 gene is activated during the intermediate phase (about 13 hours post infection) of the virus lytic cycle. The IVa2 protein is involved in both late gene activation and viral assembly (Zhang, W. and Imperiale, M. J., J Virol 74:2687-2693 (2000); Zhang et al., J Virol 75:10446-10454 (2001); Zhang et al., J Virol 77:3586-3594 2003); Tribouley et al., J Virol 68:4450-4457 (1994); Lutz et al., J Virol 70:1396-1405 (1996)). The IVa2 protein consists of 449 amino acids and spans about 1.8 kb sequence that can be divided into two exons and one intron. The entire gene can be inverted in a similar manner as the pIX gene in the viral backbone at its natural position, thereby, further eliminating the 264 bp overlap region from the vector. It is predicted that this short deletion should not affect the polymerase gene coding sequence that co-resides with the IVa2 gene.

Another approach to manipulate the 264 bp overlapping region to decrease its homology with the 293 cells is a complete or partial deletion of the 264 bp from viral gene backbone. The 264 bp portion between the AdIFNβ-RIX vector and 293 cells covers the C-terminal 83 amino acids of IVa2 protein. Gel shift assays using recombinant IVa2 proteins have demonstrated that there is a 20 amino acid amphipathic alpha helices region (in the 83 amino acid segment) that contribute to its DNA binding activity to the adenovirus major late promoter (MLP) (Lutz et al., J Virol 70:1396-1405 (1996)). Deletion of this domain from the IVa2 protein reduces (but does not abolish) its binding activity to MLP in vitro. Therefore, the 264 bp deletion may produce viable viruses. If, however, complete deletion of the 83 amino acid region results in non-viable viruses, the 20 amino acid DNA binding domain can be inserted back into its natural location. This modification will still reduce the length of the homologous region from 264 bp to about 60 bp.

Another approach to reduce the homology between the AdIFNβ-RIX vector backbone and the 293 cell genome is to replace the 83 amino acid DNA binding domain. If the 20 amino acid DNA binding domain is absolutely required for viable virus production or productive virus growth, an artificial domain can be used to replace the natural sequences using for example, alternative adenovirus serotype IVa2 domains, artificial zinc fingers or other DNA binding motifs.

All of these approaches will be useful in further reducing the homology between the AdIFNβ-RIX vector backbone and the 293 cells, to allow further reduction or complete elimination of RCA emergence.

### Heterologous Gene of Interest

In certain embodiments, the heterologous gene of interest (also referred to herein as gene of interest) is a therapeutic gene, in that the heterologous gene of interest encodes a protein that has therapeutic value or prophylactic value in a subject. In certain embodiments, the heterologous gene of interest may encode a protein or a polypeptide that is a membrane protein, such as, but not limited to, CD2, CD4, BAFF, APRIL, CD40, CD154, or an integrin protein like the α-1 integrin protein. In certain embodiments, the heterologous gene of interest may encode a protein or a polypeptide that is an intracellular protein, such as, but not limited to, a caspase, p53, herpes simplex virus thymidine kinase or retinoblastoma protein. In certain embodiments, the heterologous gene of interest may encode a costimulatory protein or a polypeptide of the immune system, such as, but not limited to, CD40L, CD27, OX-40, 4-1BB, ICOS, LIGHT, B7.1, B7.2, CD40, CD70, OX-40L, 4-1BB L, ICOS-L and HVEM. In certain embodiments, the heterologous gene of interest may encode said protein fragments thereof, said active fragments thereof, said mature forms thereof as well as proteins having substantial identity with said proteins, usually at least about 70% identity, preferably at least about 75% identity, more preferably about 80% identity, and most preferably at least about 95% identity. In certain embodiments, the heterologous gene of interest may encode a fusion or chimeric protein comprising such proteins and/or fragments.

In certain embodiments, the heterologous gene of interest may encode a protein or a polypeptide that is a secreted protein, such as, but without limitation, an interferon (IFN), such as interferon-beta, interferon-alpha and interferon-gamma, an interleukin (IL), such as IL-1, IL-2, IL-4, IL-8 and IL-12, and growth factors, such as GM-CSF and G-CSF. In a particular embodiment of this invention, the protein encoded by the heterologous gene of interest is an interferon-β gene. In another particular embodiment, the protein encoded by the heterologous gene of interest is a human interferon-β gene. In certain embodiments, the heterologous gene of interest may encode said protein fragments thereof, said active fragments thereof, said mature forms thereof as well as proteins having substantial identity with said proteins, usually at least about 70% identity, preferably at least about 75% identity, more preferably about 80% identity, and most preferably at least about 95% identity. In certain embodiments, the heterologous gene of interest may encode a fusion or chimeric protein comprising such proteins and/or fragments.

The heterologous gene of interest may encode, but is not limited to, a protein or a polypeptide that is a cytokine, a hormone, an oncogene, or a tumor suppressor gene. In certain embodiments, the heterologous gene of interest may encode said protein fragments thereof, said active fragments thereof, said mature forms thereof as well as proteins having substantial identity with said proteins, usually at least about 70% identity, preferably at least about 75% identity, more preferably about 80% identity, and most preferably at least about 95% identity. In certain embodiments, the heterologous gene of interest may encode a fusion or chimeric protein comprising such proteins and/or fragments.

The heterologous gene of interest may encode a protein or a polypeptide that is vascular endothelial factor, TNF-alpha, TNF-beta, TGF-beta, insulin-like growth factor I, insulin, Huntington's disease gene, the cystic fibrosis gene, and human growth hormone gene. In certain embodiments, the heterologous gene of interest may encode said protein fragments thereof, said active fragments thereof, said mature forms thereof as well as proteins having substantial identity with said proteins, usually at least about 70% identity, preferably at least about 75% identity, more preferably about 80% identity, and most preferably at least about 95% identity. In certain embodiments, the heterologous gene of interest may encode a fusion or chimeric protein comprising such proteins and/or fragments.

The heterologous gene of interest may encode an antibody, an antigen binding fragment thereof or an immunoreactive fragment thereof. In certain embodiments, the heterologous gene of interest may encode said protein fragments thereof, said active fragments thereof, said mature forms thereof as well as proteins having substantial identity with said proteins, usually at least about 70% identity, preferably at least about 75% identity, more preferably. about 80% identity, and most preferably at least about 95% identity. In certain embodiments, the heterologous gene of interest may encode a fusion or chimeric protein comprising such proteins and/or fragments.

The heterologous gene of interest may encode any protein or polypeptide, fragments thereof, fusion proteins or polypeptides thereof, of any gene that may have a therapeutic or prophylactic effect in a subject.

The heterologous gene of interest may encode a gene product that may be used for diagnostic purposes.

### Expression Control Elements

The adenoviral vectors described herein comprise genes that may need to be expressed at an appropriate level, time and/or in a specific cell or cells. Appropriate expression control elements need to be provided. See, e.g., Lodish et al., Molecular Cell Biology, 3^{rd} edition, W.H. Freeman and Company, New York (1995).

The appropriate expression control elements to control for the transcription and translation of a gene are well known in the art. See, e.g., Lodish et al., Molecular Cell Biology, 3^{rd} edition, W.H. Freeman and Company, New York (1995).

A gene can be under the control of a constitutive promoter or an inducible promoter. A gene under the control of a constitutive promoter is expressed under all conditions of cell growth. A constitutive promoter can be strong or weak in its ability to drive expression of a gene under its control. Exemplary constitutive promoters include the following promoters: dihydrofolate reductase (Scharfmann et al., Proc. Natl. Acad. Sci. USA 88:4626-4630 (1991)), pyruvate kinase, and β-actin (Lai et al., Proc. Natl. Acad. Sci. USA 86:10006-10010 (1989)). In addition, many viral promoters function constitutively in eukaryotic cells. Examples of such viral promoters include the early and late promoters of SV40 (Bernoist and Chambon, Nature, 290:304 (1981)), the long terminal repeats of Moloney Leukemia Virus and other retroviruses (Weiss et al., RNA Tumor Viruses, Cold Spring Harbor Laboratory, Cold Spring Harbor, N.Y. (1985)); the thymidine kinase promoter of the Herpes Simplex Virus (HSV) (Wagner et al., Proc. Nat. Acad. Sci. USA, 78:1441 (1981)); the CMV immediate-early (IE1) promoter (Karasuyama et al., J. Exp. Med., 169:13 (1989)); the promoter of the Rous sarcoma virus (RSV) (Yamamoto et al., Cell, 22:787 (1980)), and the adenovirus major late promoter (Yamada et al., Proc. Nat. Acad. Sci. USA, 82:3567 (1985)). Other suitable constitutive promoters are known in the art. In a particular embodiment of this invention, the heterologous gene of interest is placed under of the CMV immediate early promoter.

An inducible promoter may be desirable in some circumstances. For example, if the delivery of the heterologous gene of interest encoded by the therapeutic adenoviral vector is needed in particular cells, it may be desirable to target the expression of that heterologous gene of interest to those cells.

There are many promoters known in the art that are only expressed in specific cells or specific tissues. Some examples of such promoters are liver-specific promoters of hepatitis-β virus (Sandig et al., Gene Therapy, 3:1002-1009 (1996)), mammary carcinoma specific β-casein promoter, melanoma-specific tyrosinase promoter, osteosarcoma-specific c-sis promoter, and glioma and neuroblastoma-specific calcineurin A alpha promoter. Other suitable tissue specific or cell-type specific promoters are known in the art.

Many genes that are under the control of inducible promoters are active in the presence of an inducing agent. Certain inducible promoters include responsive elements (REs) which stimulate transcription when their inducing factors are bound. For example, there are REs for serum factors, steroid hormones, retinoic acid and cyclic AMP.

If the heterologous gene of interest of the adenovirus preparation of the invention is under the control of an inducible promoter, that heterologous gene of interest is triggered by exposing the genetically modified cell in vitro or in situ to conditions permitting transcription of the heterologous gene. For example, in situ expression by genetically modified cells of interferon protein encoded by an interferon gene under the control of the metallothionein promoter is enhanced by contacting the genetically modified cells with a solution containing the appropriate inducing metal ions in situ.

Systems have also been developed which allow precise regulation of gene expression by exogenously administered small molecules. These include the FK506/Rapamycin system (Rivera et al., Nature Medicine, 2(9): 1028-1032 (1996)), the tetracycline system (Gossen et al., Science, 268: 1766-1768 (1995)), the ecdysone system (No et al., Proc. Nat. Acad. Sci. USA, 93: 3346-3351 (1996)) and the progesterone system (Wang et al., Nature Biotechnology, 15:239-243 (1997)).

Enhancer DNA sequences can be inserted into the vector of the invention in order to obtain desired levels of transcriptional activity from genes encoded by said vectors of the invention. Examples of enhancers include, but are not limited to, the SV40 enhancer, which is located on the late side of the replication origin at base pair 100 to 270, the cytomegalovirus early promoter/enhancer, and adenovirus enhancers. Enhancers may work in conjunction with a promoter to regulate levels of expression and be inducible or be cell-type or tissue specific.

To express a eukaryotic gene, a poly adenylation (polyA) signal sequence, should be included. Such sequences are well known in the art. See, e.g., Sambrook et al. supra. See also Lodish et al., Molecular Cell Biology, 3^{rd} Ed., W.H. Freeman and Company, New York (1995).

Hence, by selecting the appropriate promoter (constitutive versus inducible; strong versus weak), it is possible to control both the existence and level of expression of a gene of interest.

### Adenoviral Particles

The invention also provides adenoviral particles comprising an adenoviral vector of this invention. Such adenoviral particles can be produced in the host cell lines as described below.

### Host Cell Lines

The invention provides host cell lines to complement replication-incompetent recombinant adenoviral vectors so that RCA-free replication-defective recombinant adenovirus particles can be produced in these cell lines.

Host cell lines to complement replication-incompetent recombinant adenoviral vectors of the invention may be created to support generation of replication-defective recombinant adenoviral particles.

To produce adenoviral stock (also referred to herein as “preparation”), a host cell line comprising an adenoviral vector of this invention is provided. In certain embodiments, the host cell provides in trans one or more adenoviral proteins encoded by the E1 locus.

An adenoviral stock comprising adenoviral particles of this invention comprising an adenoviral vector of this invention is provided. The production of an RCA-free adenoviral stock or preparation can be prepared and purified by any routine methods known in the art (e.g., Graham et al., Manipulation of adenovirus vectors, Vol 7, Ed. 1 (Clifton, N.J., The Humana Press Inc.)).

In certain embodiments of the invention, the host cell line of this invention is a mammalian cell line. In another embodiment, the mammalian cell line of the invention is a human cell line. In another embodiment, the human cell line is a human embryonic kidney 293 cell. Other mammalian cell lines may also be used, including, without limitation, primate cell lines and rodent cell lines. Host cell lines of the present invention may consist of a variety of cell types, such as fibroblasts or epithelial cells.

The 293 human embryonic kidney cell line is a suitable cell line for use as a host cell line. 293 cells have been demonstrated to allow replication of adenoviruses that lack the adenoviral E1 gene products (Graham et al. (1997) J. Gen. Virol. 36:59-74) by providing them in trans to support adenovirus replication.

In certain embodiments, the host cell genome and the adenoviral genome have overlapping sequences of no more than 280 bp in the right-arm region. In a particular embodiment, the overlapping sequences are no more than 264 bp in the right-arm region.

To create a host cell line comprising an adenoviral vector of this invention, the adenoviral vector is introduced into the host cell by any method known in the art. In general, the adenoviral vector of the invention can be introduced into a cell by calcium phosphate transfection (see, e.g., Methods in Molecular Biology, Vol. 7, Gene Transfer and Expression Protocols, Ed. E. J. Murray, Humana Press (1991)), DEAE-dextran mediated transfection (see, e.g., Methods in Molecular Biology, Vol. 7, Gene Transfer and Expression Protocols, Ed. E. J. Murray, Humana Press (1991)), cationic liposome-mediated transfection(se e, e.g., Methods in Molecular Biology, Vol. 7, Gene Transfer and Expression Protocols, Ed. E. J. Murray, Humana Press (1991)), microinjection infection, or electroporation (Brash et al., Molec. Cell. Biol. 7: 2031-2034 (1987)). Such methods are described in many standard laboratory manuals, such as Sambrook et al., supra, Ausubel et al., supra and Davis et al., Basic Methods in Molecular Biology, McGraw-Hill, 1^{st} Ed., New York (1986).

Transfection of cells refers to the acquisition by a cell of new genetic material by incorporation of added DNA.

Infection of cells refers to infection of cells by wild type or modified adenovirus. This process is also referred to as “transduction of cells”.

The term “transformation” as used herein refers to any genetic modification of cells and includes both “transfection” and “transduction”.

A host cell line, comprising an adenoviral vector of this invention, can be selected for or screened for, or both. For example, the presence of the protein product(s) encoded by the heterologous gene of interest on the adenoviral vector could be tested by one of ordinary skill in the art. For instance, if the heterologous gene of interest is human interferon-β, then western blotting for interferon-β protein verifies that the host cell line of the invention contains the therapeutic adenoviral vector. Also, the presence of the therapeutic vector can also be confirmed by testing for the presence of other adenoviral gene products not found in the host cell line without the therapeutic adenoviral vector. If the heterologous gene encoded by the adenoviral vector of this invention is a reporter gene, such as GFP or lacZ, expression of the marker gene may be monitored, by methods well known in the art. See Sambrook et al. and Ausubel et al., supra. PCR may be used to test for the presence of an adenoviral vector of the invention by designing primers that will amplify a product that would not be found in a cell that does not contain an adenoviral vector of the invention and that one of ordinary skill in the art could easily design such primers and carry out such an analysis.

The presence of an adenoviral vector of this invention in the host cell can also be confirmed by replication of the therapeutic adenoviral vector, by assays such as the cytopathic effect (CPE) assay.

For example, a CPE assay may be performed as follows. Twenty-four hours prior to transfection, host cells comprising a complementing vector are plated at, for example, 1−2×10⁶ cells per 60 mm culture plate. The cells should be 50-70% confluent at the time of transfection. Using a standard transfection method, the adenoviral vector DNA is introduced into the host cells. The cells are returned back to the 37° C./5% CO₂ tissue culture incubator. The cells should be periodically observed for cytopathic effect (CPE). As known to one skilled in the art, CPE appearance (holes or plaques where there are no cells in a lawn of cells) depends on transfection efficiency and can take up to two weeks to be visible, depending on the type of host cell line used. Upon observation of CPE, cells are harvested and subjected to a series of freeze-thaw cycles to obtain adenovirus still associated with the host cells. Adenovirus may be further amplified to achieve a higher titer by further infecting host cells with the adenovirus obtained with the above-identified steps.

As another example, the production of adenovirus particles, which also confirms the presence of the adenoviral vectors of this invention in the host cells, can be ascertained by assays, such as the plaque formation assay, which can also determine virus titer. Other assays known in the art to determine virus production and titer include an end-point dilution assay and an assay that measures optical density at wavelength 260 nm. It may be advisable to use two different assays to confirm the viral titer of a particular adenoviral preparation.

For example, a plaque formation assay may be performed as described briefly below. Twenty-four hours prior to infection, appropriate host cells (e.g., 293 cells) that can complement the adenoviral vector of this invention are plated at a density of 0.5-1×10⁵ cells per well of a six-well tissue culture plate in appropriate cell culture media. The adenoviral preparation or composition for which the titer is to be determined is diluted. An exemplary range of dilutions to be tested is 10⁻⁵ to 10⁻¹⁰. Prior to infection with the diluted adenoviral preparations or composition, the cells in the wells of the six-well plate should be 80-90% confluent. The cell culture media is removed from the cells, the diluted adenovirus is added, and the plates are carefully tipped to spread the diluted virus over the cells. The cell culture plates are returned to the 37° C./5% CO₂ tissue culture incubator for a period of approximately one hour. Following this one hour, the cells are overlaid with a cell culture media solution containing a percentage of agarose. For example, a 5% agarose-cell culture media solution can be used. Other solutions containing cell culture media, nutrients, and agarose for the cells being overlaid are known in the art. The agarose-cell culture media solution chosen is carefully added without dislodging the cells of each well of the six-well tissue culture plates containing infected cells. After the agarose has solidified and set, the tissue culture plates are returned to the tissue culture incubator. Plaques, or clear areas where there are no cells, should be visible in 7 to 10 days and can be counted for each dilution. In general, 3 to 4 wells should be plated with cells for each dilution of the adenoviral preparation or composition tested. The viral titer is calculated using the following equation: # plaques/(d×V)=pfu/ml, where “d” stands for dilution factor and “V” stands for the volume of diluted adenoviral preparation added to the well.

Also, the adenovirus particles produced in a host cell line comprising an adenoviral vector of this invention should not have, or should have few particles with the adenoviral genomic sequences not on the therapeutic vector. This can be tested by, for example, PCR. For instance, if the adenoviral vector lacks the E1 genes, PCR can be used to confirm that the adenovirus produced in this cell line lacks such genes. PCR experiments can also confirm the presence of the replication-incompetent adenovirus in the adenoviral preparations of this invention. PCR experiments can further be used to confirm that the adenovirus particles produced in the host cell line comprises an adenoviral vector in which the pIX gene is in an inverted orientation relative to the direction of transcription of the native pIX gene at a location where the pIX gene normally resides.

As shown in Example 1, a host cell line of this invention was generated by transfecting 293 cells with an adenoviral vector comprising the inverted pIX gene at its natural location whose expression is directed by its natural pIX promoter. The presence of the adenoviral vector in the host 293 cells was confirmed by using the CPE assay and the plaque formation assay, as described above. Example 1 also demonstrates that such host cell lines of the invention were able to produce adenoviral particles comprising the adenoviral AdIFNβ-RIX vector of the invention, as indicated by the detection of human IFNβ expression in the cell lysates.

### Production of RCA-Free Adenoviral Preparations

This invention provides RCA-free, or substantially RCA-free, adenoviral preparations (also referred to herein as adenoviral stock). This invention also provides methods for propagating RCA-free adenoviral particles.

In certain embodiments, the invention provides a method of propagating RCA-free adenoviral preparations comprising adenoviral particles comprising the steps of introducing an adenoviral vector of this invention described in detail herein into a host cell; culturing said host cell and recovering said adenovirus particles from said cell culture; and optionally, purifying said recovered viral particles.

In certain embodiments, an adenovirus preparation of this invention thus comprises recombinant adenovirus purified from host cells of this invention, which host cell comprises an adenoviral vector of this invention and which host cell used is the human embryonic kidney 293 cell. In certain embodiments, the recombinant adenovirus comprises an adenoviral genome in which the pIX gene is in an inverted orientation relative to the direction of transcription of the native pIX gene at a location where the pIX gene normally resides and wherein said inverted pIX gene is placed under the control of the pIX natural promoter. In certain embodiments, this adenoviral vector further comprises a deletion of all or a part of the IVa2 gene, replacement of a part or all of the IVa2 gene with another domain (as described herein) or an inversion of the IVa2 gene at the location where the IVa2 gene normally resides.

To make the recombinant adenovirus from host cells transfected with an adenoviral vector of this invention, the recombinant adenovirus is purified from cell lysate from a host cell of this invention comprising an adenoviral vector. Suitable routine purification methods may be used, some of which are described below.

Host cells comprising the adenoviral vector of this invention are lysed and the lysate from the cells comprising the adenoviral vector is collected. The lysate comprises adenovirus particles. Cells can be lysed by any method known in the art, such as freeze-thawing, autolysis, hypotonic or hypertonic solution lysis, sonication, and detergent lysis. One such lysis/collection method is detailed as follows. The cells are subjected to two or three rounds of freeze-thawing, the resulting lysate is subjected to centrifugation to remove cellular debris, and the lysate is collected. The recombinant adenovirus particles are purified from the cell lysate by conventional purification techniques, such as density gradient centrifugation (on, for example, a cesium chloride gradient), affinity chromatography (such as with a resin that is derivatized with antibodies specific to capsid proteins), ion exchange chromatography or other chromatographic techniques (such as size exclusion or hydrophobic interaction chromatography), or a combination of purification techniques. It is also possible to prepare and purify adenovirus particles from the supernatant of cells bearing the adenoviral vectors. The supernatant is simply collected after subjecting the cells to centrifugation. The virus in the supernatant may then be purified, as described above.

As shown in Example 1, such RCA-free adenoviral preparations are prepared by purifying the virus clones which comprise an adenoviral vector of the invention and amplifying said adenoviruses before harvesting and purification.

Verification of the absence or minimal existence of RCA in adenoviral preparations or compositions of this invention can be accomplished by, e.g., polymerase chain reaction (PCR) methodology, such as by ascertaining the absence of the E1 gene sequences. As shown in Example 1, viral genomic DNA from adenoviral particles produced from 293 cells was subjected to real time PCR amplification for the presence of the E1 sequences. No E1 gene sequences were detectable in the viral DNA of viral particles comprising an adenoviral vector of the invention, indicating that no RCA existed in the adenoviral preparation of the invention detailed in Example 1.

Verification of the absence or minimal existence of RCA in adenoviral preparations or compositions of this invention can also be accomplished by, e.g., liquid culture assay. This assay is less sensitive than the PCR methodology but it is more reliable in determination of infectious units, as described below. It involves a three-step procedure in which the adenoviral vector is first subjected to absorption on HeLa cells. Next, any putative RCA would be transferred to A549 cells followed by culturing to amplify any replicative recombinants. Finally, culture supernatants were assayed on naive A549 cells to verify presence of replicative virions. As shown in Example 1, the liquid culture assay confirmed the real-time PCR results by indicating the absence of RCA in the adenoviral particles comprising an adenoviral vector of this invention.

Demonstration of the absence or minimal existence of RCA in adenoviral preparations or compositions of this invention can be accomplished by serially passaging cells for RCA generation. Two approaches can be used to serially passage cells in order to verify the absence of RCAs. One approach involved inoculating cells with the adenoviral vector of the invention followed by harvesting upon detection of cytopathic effects. The cells were pelleted, followed by three cycles of freeze/thaw to generate crude viral lysates (“CVL”). Naive cells were inoculated with a dilution of the CVL and cells were passaged for 10 passages in order to detect RCA emergence. The second approach involved large scale serial passaging of cells in cell factories using the same techniques as described above.

### System

This invention also provides a system for generating RCA-free, or substantially RCA-free, adenovirus preparations. In certain embodiments, the invention provides a system comprising a host cell which complements in trans a deficiency in one or more essential gene functions of the E1 region of an adenoviral genome, and an adenoviral vector of this invention, such as one comprising an adenoviral genome in which the pIX gene is in an inverted orientation relative to the direction of transcription of the native pIX gene at a location where the pIX gene normally resides and wherein said inverted pIX gene is placed under the control of the pIX natural promoter. In certain embodiments, this adenoviral vector further comprises a deletion of all or a part of the IVa2 gene, replacement of a part or all of the IVa2 gene with another domain (as described herein) or an inversion of the IVa2 gene at the location where the IVa2 gene normally resides. In certain embodiments, the system provides that there is insufficient overlapping sequences between said host cellular genome and said adenoviral genome to appreciably mediate a recombination event sufficient to result in a replication-competent adenovirus.

### Compositions

This invention also provides compositions, such as pharmaceutical compositions, comprising an adenoviral preparation or adenovirus particle of this invention. These compositions thus comprise purified adenovirus particles.

### Formulations

An adenoviral composition or preparation of the invention is administered in a pharmaceutically effective, prophylactically effective or therapeutically effective amount, which is an amount sufficient to produce a detectable, preferably medically beneficial effect in a subject suffering from, or at risk of suffering from a disease, a disorder or a condition amenable to gene therapy.

The composition or adenovirus preparation of this invention can be in any suitable form, depending on the route of administration selected and the disease or disorder that needs to be treated or prevented.

One or more adenoviral compositions (including, but not limited to pharmaceutical compositions) or preparations of the invention could be administered to a subject alone or with a pharmaceutically acceptable carrier. As used herein, “pharmaceutically acceptable carrier” means any and all solvents, dispersion media, coatings, antibacterial and antifungal agents, isotonic and absorption delaying agents, and the like that are physiologically compatible. Some examples of pharmaceutically acceptable carriers are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition or preparation. Additional examples of pharmaceutically acceptable substances are wetting agents or minor amounts of auxiliary substances such as wetting or emulsifying agents, preservatives or buffers, which enhance the shelf life or effectiveness of the composition of the invention. Acceptable carriers include biocompatible, inert or bioabsorbable salts, buffering agents, oligo- or polysaccharides, polymers, viscoelastic compound such as hyaluronic acid, viscosity-improving agents, preservatives, and the like.

The resulting therapeutic composition, suspended, dissolved or dispersed in a pharmaceutically acceptable carrier or excipient, should not adversely affect the recipient's homeostasis, particularly electrolyte balance. Thus, an exemplary carrier comprises normal physiologic saline (0.15M NaCl, pH 7.0 to 7.4). Other acceptable carriers are well known in the art and are described, for example, in Remington's Pharmaceutical Sciences, Gennaro, ed., Mack Publishing Co., (1990), herein incorporated by reference.

In certain embodiments, the adenoviral compositions or preparation of the present invention may be prepared with a carrier that will protect the adenovirus preparations or composition against rapid release, such as but not limited to, a controlled release formulation, including implants, and microencapsulated delivery systems. See, e.g., Sustained and Controlled Release Drug Delivery Systems (J. R. Robinson, ed., Marcel Dekker, Inc., New York, 1978), herein incorporated by reference.

### Treatment And Prevention Of Disease, Disorder or Condition

The adenovirus preparations or compositions of the present invention can be used to treat a subject (i.e. a patient) in need thereof via gene therapy, including in vivo or ex vivo gene therapy.

A subject in need of gene therapy is a subject suffering from a disease, disorder or condition that can be treated or prevented by administering an adenoviral preparation or a composition of this invention, or by administering cells from the subject extracted from the subject and treated with an adenoviral preparation or a composition of this invention. That subject may be a mammalian subject. A preferred subject is a human subject.

A disease, a disorder or a condition amenable to gene therapy can be any disease, a disorder or a condition amenable to gene therapy.

A disease, a disorder or a condition amenable to gene therapy includes cancer, precancerous conditions, and genetic disease or condition such as muscular dystrophy. A disease, a disorder or a condition amenable to gene therapy includes conditions such as genetic diseases (i.e., a disease condition that is attributable to one or more gene defects), acquired pathologies (i.e., a pathological condition that is not attributable to an inborn defect) and prophylactic processes (i.e., prevention of a disease or of an undesired medical condition). An acquired pathology may be a disease or syndrome manifested by an abnormal physiological, biochemical, cellular, structural or molecular biological state.

A disease, a disorder or a condition amenable to gene therapy may be an infection, including viral and bacterial infection, a hyperproliferative disease or disorder, including cancer and pre-cancerous conditions, genetic immunodeficiency conditions, such as hyper-IgM syndrome, and primary or combined immunodeficiency conditions, including conditions characterized by neutropenia.

A disease, a disorder or a condition amenable to gene therapy may be hyperproliferative diseases or disorders, including cancers. Such diseases or disorders can involve any cells, tissue or organ, including brain, lung, squamous cell, bladder, stomach, pancreas, breast, head, neck, liver, kidney, ovary, prostate, colon, rectum, esophagus, nasopharynx, thyroid and skin. The cancer may be melanoma, lymphoma, leukemia, multiple myeloma, sarcoma or carcinoma. The cancer may be solid tumors or may involve a bodily fluid, such as blood.

A disease, a disorder or a condition amenable to gene therapy may be genetically inherited diseases, such as Huntington's disease, bipolar disorder, Parkinson's disease, Carpal Tunnel Syndrome, cystic fibrosis, Pelizaeus-Merzbacher Disease, multiple sclerosis or Duchenne Muscular Dystrophy.

A disease, a disorder or a condition amenable to gene therapy may be an infectious disease, such as tuberculosis, malaria, yellow fever, or a disease caused by infection by hepatitis B virus, herpesviruses, human immunodeficiency virus, etc.

Diseases or conditions that are amenable to gene therapy and could be treated by interferon-β gene therapy include, but are not limited to, a viral infection such as hepatitis B, C or D virus infection, human papilloma virus infection, herpes simplex virus infection, herpes zoster virus infection, cytomegalovirus (CMV) infection, HIV infection or rhinovirus infection; a cancer such as hemangioma, glioma, ovarian cancer, breast cancer, leukemia, mesothelioma, colorectal cancer or inoperable non-small cell lung carcinoma. See, e.g., PCT publication WO 99/10516, published Mar. 4, 1999.

Prevention/treatment of a disease with a heterologous gene encoding a secreted protein requires lower amount of adenoviral composition or preparations. See, e.g., PCT publication WO 99/10516, published Mar. 4, 1999.

In some embodiments, adenoviral preparations or compositions of the invention can be used as a vaccine or as adjuvants to a vaccine.

In certain embodiments, the heterologous gene encoded by the replication-defective adenovirus in the composition or preparation of this invention is expressed in one or more of the cells in the subject. The heterologous gene may be expressed in the cells removed from a subject and exposed to an adenoviral preparation or composition of this invention. The levels of expression of the heterologous gene can be monitored by taking a sample from the treated patient or from the subject's cells cultured in vitro, and using an assay to detect expression of that protein. Detection of expression depends on the protein in question.

The compositions of this invention may be in a variety of forms. For example, they may be in liquid, semi-solid and solid dosage forms, such as liquid solutions (e.g., injectable and infusible solutions), dispersions or suspensions, or liposomes. The preferred form depends on the intended mode of administration and therapeutic application. Typical preferred compositions are in the form of injectable or infusible solutions.

Therapeutic compositions typically should be sterile and stable under the conditions of manufacture and storage. The composition can be formulated as a solution, microemulsion, dispersion, liposome, or other ordered structure suitable to high drug concentration. Sterile injectable solutions can be prepared by incorporating the adenovirus preparation or composition of the invention in the required amount in an appropriate solvent with one or a combination of ingredients enumerated above, as required, followed by filtered sterilization. Generally, dispersions are prepared by incorporating the adenovirus preparation or composition of the invention into a sterile vehicle that contains a basic dispersion medium and the required other ingredients from those enumerated above. Prolonged absorption of injectable compositions can be brought about by including in the composition an agent that delays absorption, for example, monostearate salts and gelatin. The preparations of this invention may be administered at or near the same time in conjunction with another therapeutic, diagnostic or prophylactic agent.

### In Vivo Gene Therapy

This invention relates to a method of treating a disease or condition in a subject or preventing a subject from developing a disease or condition comprising the step of administering an RCA-free, or substantially RCA-free, adenovirus preparation of this invention or a composition comprising an RCA-free, or substantially RCA-free, adenovirus preparation of this invention. The disease or disorder to be treated or prevented from developing can be any disease or disorder suitable for treatment or prevention by gene therapy. In certain embodiments, the adenovirus preparation of this invention or a composition comprising an adenovirus preparation of this invention is administered to a subject by a route such as local administration, intraocular administration, parenteral administration, intranasal administration, intratracheal administration, intrabronchial administration, intramuscular administration, intravenous administration, intraperitoneal administration or subcutaneous administration. In certain embodiments, the adenovirus preparation of this invention or a composition comprising an adenovirus preparation of this invention is injected into a subject locally, (such as by direct injection into a tumor and/or near a tumor, if the disease is cancer) or at a site distant from the diseased tissue or cells.

In vivo gene therapy is an especially advantageous method by which to treat diseases, disorders or conditions amenable to gene therapy with a heterologous gene encoding a secreted protein. Accordingly, in one embodiment of this invention, the heterologous gene encoded by a therapeutic adenoviral vector encodes a secreted protein, such as interferon-β. Delivery of a secreted protein by recombinant adenoviral preparations in gene therapy is an efficient method by which one can deliver a therapeutic protein, as such delivery generally requires a smaller amount of the adenoviral preparation or composition. See, e.g., PCT publication WO 99/10516, published Mar. 4, 1999.

As shown in Example 2, an inverted pIX-containing adenoviral vector encoding human IFNβ of this invention displayed tumor killing activity in a xenograft tumor model in nude mice, suggesting that the modification of the pIX gene did not affect the human IFNβ expression levels or the tumor killing capability.

In certain other embodiments, another heterologous gene that may be encoded by a therapeutic adenoviral vector of the present invention is LIGHT, which is a costimulatory protein of the immune system that has been associated with an enhancement in the tumor antigen recognition process. LIGHT is a new molecular entity with a novel function of breaking existing stromal tumor barrier (Mauri et al., Immunity 8:21-30 (1998); Zhai et al., J. Clin. Investigation 102:1142-1151 (1998); U.S. Pat. No. 6,955,883). In addition to its immune-stimulating properties, LIGHT also up-regulates a panel of cytokines and chemokines (including IFNγ, GM-CSF and SLC) which are being explored as anti-tumor therapies.

Boosting the immune system of cancer patients to eradicate primary tumor and prevent metastasis at distal sites has been an attractive approach in cancer therapy. A number of immune stimulants and strategies have been tested in clinical trials (Houghton, A. N., Nat Immunol 5:123-124 (2004); Rosenberg et al., Nat Med 10:909-915 (2004)). One approach involves the over-expression of co-stimulation molecules to enhance tumor antigen recognition process. Therefore, direct LIGHT gene delivery into tumors using an adenoviral vector of the present invention is contemplated as a novel approach for cancer immunotherapy.

Because of the strong adjuvant effects of the adenoviral capsid and its nature of transient gene expression, LIGHT gene delivery into tumors using an adenoviral vector of the present invention is predicted to be superior to other means of delivery. Direct gene delivery of LIGHT avoids the tedious process of the “tumor vaccine” approach involving cell isolation and ex vivo transduction. In addition, tumor cells will be preferentially transduced when an adenoviral vector of the present invention expressing LIGHT is administered intratumorally because selective transduction of tumor cells, and not T cells, using an adenoviral vector of the present invention will minimize unwanted side effects and systematic toxicity, such as the development of autoimmune diseases as seen in transgenic mice expressing LIGHT.

In certain embodiments, the heterologous gene that may be encoded by a therapeutic adenoviral vector of the present invention is a mutant version of LIGHT known as mutant LIGHT, as described in U.S. publication 20050025754 and International publication WO 2005/002628. Specifically, mutant LIGHT is generated to prevent protease digestion so that LIGHT can be expressed on tumor cells. Mutant LIGHT has the proteolytic site EKLI from positions 79-82 deleted from the amino acid sequence of native LIGHT and has been shown to be useful for eliciting high levels of chemokines and adhesion molecules, accompanied by massive infiltration of naive T lymphocytes.

Furthermore, the ability of adenoviral gene delivery of LIGHT or mutant LIGHT to stimulate cellular immunity via its up-regulation of cytokines and chemokines can be very powerful in eliminating micrometastatic tumors, suppressing dormant tumor sites and killing residue tumor cells. Thus, it is anticipated that LIGHT gene delivery can be used in combination with surgical tumor debulking procedures, radiation therapy or other chemotherapies.

### Vaccines

Adenoviral vectors has been extensively studied as a potential vaccine platform because of its ability to induce potent cellular and humoral immunity. Data from animal models have suggested that adenoviral vectors are effective in protecting against infections caused by HIV, herpes simplex virus and Mycobacterium tuberculosis (Barouch et al., Hum Gene Ther 16:149-156 (2005); Seth P. Cancer Biol Ther 4:512-517 (2005)). However, as described in U.S. Pat. No. 6,733,993, the replication-defective adenoviral vectors described in that patent, allegedly suitable for use in gene therapy as HIV vaccines, are at least partially deleted in E1 and comprises a wildtype adenovirus cis-acting packaging region from about base pair 1 to between about base pair 342 to about 458 (preferably, 1-450) and, preferably, base pair 3511-3523 to about base pair 5798 of a wild-type adenovirus sequence. Thus, these adenoviral vectors still possess substantial sequence homology (approximately 450 bp and over 1 kb) between the integrated adenoviral sequences in the cell (such as 293 and PER.C6. cells) and the viral sequences on both sides of the E1 deletion in the vector. Because of the extensive sequence homology, homologous recombination events leading to the generation of RCA is still highly probable using this adenoviral vector system.

Thus, it is contemplated that the adenoviral vectors of the present invention can be used as effective vaccines for the treatment of diseases. Accordingly, this invention provides for a vaccine composition comprising a recombinant replication-defective adenoviral vector of this invention comprising a heterologous gene of interest that can be used as a vaccine after administering this vector to a subject.

In certain embodiments, the recombinant adenoviral vector comprises a heterologous gene of interest, which is a non-adenoviral gene. This heterologous gene encodes a gene product, which may be a protein, or an RNA. The intended use of a vaccine for the resulting adenoviral preparation or composition dictates the nature of the heterologous gene of interest encoded by the adenoviral vector.

In certain embodiments, the heterologous gene of interest may encode a protein or a polypeptide that is a membrane protein, such as, but not limited to, CD2, CD4, BAFF, APRIL, CD40, CD154, or an integrin protein like the α-1 integrin protein. In certain embodiments, the heterologous gene of interest may encode a protein or a polypeptide that is an intracellular protein, such as, but not limited to, a caspase, p53, herpes simplex virus thymidine kinase or retinoblastoma protein. In certain embodiments, the heterologous gene of interest may encode a costimulatory protein or a polypeptide of the immune system, such as, but not limited to, CD40L, CD27, OX-40, 4-1BB, ICOS, LIGHT, B7.1, B7.2, CD40, CD70, OX-40L, 4-1BB L, ICOS-L and HVEM. In certain embodiments, the heterologous gene of interest may encode said protein fragments thereof, said active fragments thereof, said mature forms thereof as well as proteins having substantial identity with said proteins, usually at least about 70% identity, preferably at least about 75% identity, more preferably about 80% identity, and most preferably at least about 95% identity. In certain embodiments, the heterologous gene of interest may encode a fusion or chimeric protein comprising such proteins and/or fragments.

In certain embodiments, the heterologous gene of interest may encode a protein or a polypeptide that is a secreted protein, such as, but without limitation, an interferon (IFN), such as interferon-beta, interferon-alpha and interferon-gamma, an interleukin (IL), such as IL-1, IL-2, IL-4, IL-8 and IL-12, and growth factors, such as GM-CSF and G-CSF. In a particular embodiment of this invention, the protein encoded by the heterologous gene of interest is an interferon-β gene. In another particular embodiment, the protein encoded by the heterologous gene of interest is a human interferon-β gene. In certain embodiments, the heterologous gene of interest may encode said protein fragments thereof, said active fragments thereof, said mature forms thereof as well as proteins having substantial identity with said proteins, usually at least about 70% identity, preferably at least about 75% identity, more preferably about 80% identity, and most preferably at least about 95% identity. In certain embodiments, the heterologous gene of interest may encode a fusion or chimeric protein comprising such proteins and/or fragments.

The heterologous gene of interest may also encode, but is not limited to, a protein or a polypeptide that is a cytokine, a hormone, an oncogene, or a tumor suppressor gene. In certain embodiments, the heterologous gene of interest may encode said protein fragments thereof, said active fragments thereof, said mature forms thereof as well as proteins having substantial identity with said proteins, usually at least about 70% identity, preferably at least about 75% identity, more preferably about 80% identity, and most preferably at least about 95% identity. In certain embodiments, the heterologous gene of interest may encode a fusion or chimeric protein comprising such proteins and/or fragments.

The heterologous gene of interest may also encode a protein or a polypeptide that is vascular endothelial factor, TNF-alpha, TNF-beta, TGF-beta, insulin-like growth factor I, insulin, Huntington's disease gene, the cystic fibrosis gene, and human growth hormone gene. In certain embodiments, the heterologous gene of interest may encode said protein fragments thereof, said active fragments thereof, said mature forms thereof as well as proteins having substantial identity with said proteins, usually at least about 70% identity, preferably at least about 75% identity, more preferably about 80% identity, and most preferably at least about 95% identity. In certain embodiments, the heterologous gene of interest may encode a fusion or chimeric protein comprising such proteins and/or fragments.

The heterologous gene of interest may encode an antibody, an antigen binding fragment thereof or an immunoreactive fragment thereof. In certain embodiments, the heterologous gene of interest may encode said protein fragments thereof, said active fragments thereof, said mature forms thereof as well as proteins having substantial identity with said proteins, usually at least about 70% identity, preferably at least about 75% identity, more preferably about 80% identity, and most preferably at least about 95% identity. In certain embodiments, the heterologous gene of interest may encode a fusion or chimeric protein comprising such proteins and/or fragments.

The heterologous gene of interest may encode any protein or polypeptide, fragments thereof, fusion proteins or polypeptides thereof, of any gene that may have an effect as a vaccine in a subject.

In certain embodiments, the vaccine composition can be administered alone or in combination with other therapeutics genes, such as those discussed above.

### Route of Administration

The adenoviral compositions, preparations and vaccine compositions of the present invention can be administered by a variety of methods known in the art. For many therapeutic applications, the route of administration may be subcutaneous, intramuscular, intravenous, or intraperitoneal administration. As will be appreciated by one skilled in the art, the route and/or mode of administration will vary depending upon which disease or condition the subject has or is about to have and by whether the administration is a composition, an adenovirus preparation or cells, in case of ex vivo gene therapy.

In certain embodiments, the composition or adenovirus preparation of this invention is administered by one or more of the following routes: local administration, intraocular administration, parenteral administration, intranasal administration, intratracheal administration, intrabronchial administration, intramuscular administration, intravenous administration, intraperitoneal administration, and subcutaneous administration. The route of administration is not limited to these routes. The route of administration is selected according to the disease or condition and is routine in the art.

In the case of cancer, it may be desirable to administer the adenoviral preparation, composition, vaccine composition or cells exposed thereto by direct injection into or near the tumor.

The cells exposed in vitro to an adenoviral composition, preparation and vaccine composition of the present invention can be administered by a variety of methods known in the art. The route of administration is selected according to the disease or condition and is routine in the art. For many therapeutic applications, the route of administration for ex vivo gene therapy may be, for example, intravenous administration, intraperitoneal administration, or surgical implantation.

### Dosage

The adenoviral compositions, preparations and vaccine compositions of the invention may include a “therapeutically effective amount,” a “prophylactically effective amount” or a “diagnostically effective amount” of an adenoviral composition, preparation and vaccine composition of the present invention.

A “therapeutically effective amount” refers to an amount effective, at dosages and for periods of time necessary, to achieve the desired therapeutic result.

A “prophylactically effective amount” refers to an amount effective, at dosages and for periods of time necessary, to achieve the desired prophylactic result.

A “diagnostically effective amount” refers to an amount effective to achieve the desired diagnosis.

A therapeutically, prophylactically or diagnostically effective amount of the adenovirus preparation, composition or vaccine composition of the invention may vary according to factors such as the disease state, age, sex, and weight of the individual, and the ability of the adenovirus composition or preparation of the invention to elicit a desired response in the individual. A therapeutically, prophylactically or diagnostically effective amount is also one in which any toxic or detrimental effects of adenoviral pharmaceutical compositions or preparations of the present invention are outweighed by the therapeutically or prophylactically beneficial effects.

Dosage regimens can be adjusted to provide the optimum desired response (e.g., a therapeutic or prophylactic response). For example, a single dose can be administered, several divided doses can be administered over time or the dose can be proportionally reduced or increased as indicated by the exigencies of the therapeutic or prophylactic situation. Dosage unit form as used herein refers to physically discrete units suited as unitary dosages for the mammalian subjects to be treated; each unit containing a predetermined quantity of adenovirus composition or preparation of this invention and/or active, therapeutic compound calculated to produce the desired therapeutic effect in association with the required pharmaceutical carrier.

Suitable doses of the adenovirus preparations, compositions or vaccine compositions of this invention may be readily determined by one of ordinary skill in the art, and will depend primarily upon factors such as, but not limited to, the condition being treated, the health, age and weight of the subject, and may thus vary among subjects. An effective does may be determined by one skilled in the art and can be in the range of 0.1 to 100 ml of saline or another acceptable carrier containing from 10⁷ to about 10¹⁸ virus particles per dose, about 10⁸ to about 10¹⁷, about 10⁹ to about 10¹⁶, about 10¹⁰ to 10¹⁵, about 10¹¹ to 10¹⁴, or about 10¹² to 10¹³ viral particles per dose. In certain embodiments doses include about 10⁷, about 10⁸, about 10⁹, about 10¹⁰, about 10¹¹, about 10¹², about 10¹³, about 10¹⁴, about 10¹⁵, about 10¹⁶, about 10¹⁷, about 10¹⁸ virus particles per dose. The dose may be administered daily, weekly, monthly, bi-monthly, or at other selected intervals, as determined by one skilled in the art. Doses administered may vary with the type and severity of the condition to be treated. It is to be further understood that for any particular subject, specific dosage regimens could be adjusted over time according to the individual need and the professional judgment of the person administering or supervising the administration of the compositions or preparations, and that dosage ranges set forth herein are exemplary only.

Another consideration in determining dosage for a patient is patient convenience. While frequent injections may be necessary, a single administration, or a few infrequent administrations, of an adenoviral preparation or composition of the present invention could provide long-term stable production of the protein expressed by the heterologous gene of the therapeutic vector (now encoded by the adenoviral vector of the adenoviral composition or preparation of this invention). There exists the possibility that with repeated administration a humoral immune response might result that would limit the effectiveness of further administrations of the adenovirus preparation, composition or vaccine composition. In this instance, an immunosuppressive agent, for example, cyclosporin or an antibody directed against CD154, can be administered along with said adenoviral preparation, composition or vaccine composition.

Suitable doses of the cells exposed in vitro to adenoviral preparations, compositions or vaccine compositions of this invention for ex vivo therapy may be readily determined by one of ordinary skill in the art, and will depend primarily upon factors such as, but not limited to, the condition being treated, the health, age, and weight of the subject.

Furthermore, suitable dose of the adenoviral composition, adenovirus preparation or vaccine composition of this invention for infecting the removed cells in vitro needs to be determined. That determination is routine in the art. For example, an MOI should be used that is the lowest virus to cell ratio which will lead to expression of the gene a desired number of cells in the population to be treated prior to returning the cells to the subject. An effective dose, to be administered to the subjects' cells prior to returning the cells to the subject, may be determined by one skilled in the art and can be in the range of 0.1 to 100 ml of saline or another acceptable carrier containing from 10⁷ to about 10¹⁸ virus particles per dose, about 10⁸ to about 10¹⁷, about 10⁹ to about 10¹⁶, about 10¹⁰ to 10¹⁵, about 10¹¹ to 10¹⁴, or about 10¹² to 10¹³ viral particles per dose. In certain embodiments doses include about 10⁷ about 10⁸, about 10⁹, about 10¹⁰, about 10¹¹, about 10¹², about 10¹³, about 10¹⁴, about 10¹⁵, about 10¹⁶ about 10¹⁷, or about 10¹⁸ virus particles per dose.

### Subjects

An embodiment of the current invention is to administer an adenoviral composition, preparation or vaccine composition of this invention to a subject. In certain embodiments of the invention, the subject is a mammalian subject, such as a mouse, rat, or a primate. In certain embodiments, the primate is a human. In another embodiment of the invention, a subject is a non-human mammal.

### Biological Deposits

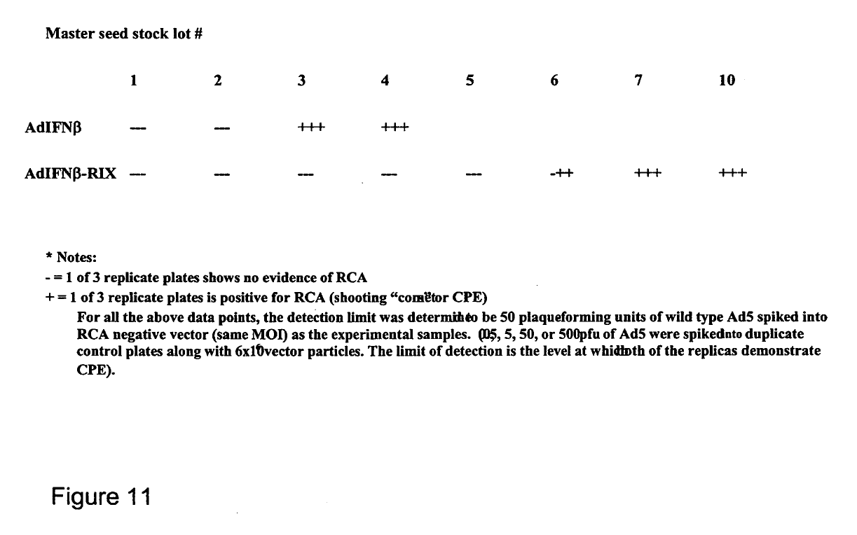

The following recombinant adenovirus H5R9CMVIFNbeta (or AdIFNβ-RIX), having ATCC Accession No. PTA-6198, was deposited with the American Type Culture Collection (“ATCC”), P.O. Box 1549, Manassa, Va. 20110-2209, U.S.A., on Sep. 10, 2004. The ATCC is located at 10801 University Blvd, Manassas, Va. 20110-2209, U.S.A. Recombinant adenovirus H5R9CMVIFNbeta comprises the adenoviral vector H5R9CMVIFNbeta (or AdIFNβ-RIX). See FIG. 11.

In order that this invention may be better understood, the following examples are set forth. These examples are for purposes of illustration only and are not to be construed as limiting the scope of the invention in any manner.

All references cited herein are hereby incorporated by reference.

### EXAMPLE I

### Construction of the AdIFNβ-RIX Plasmid Vector

Two approaches were undertaken to invert the pIX gene in the parental adenoviral vector (AdIFNβ), of which the second approach resulted in a more successful creation of AdIFNβ-RIX (this represents the AdIFNβ vector with an inverted pIX gene).

The first pIX gene inversion strategy involved the use of an internal ribosomal entry sequence (“IRES”). The use of IRES will allow coupling of pIX expression to that of the pIVa2 gene.

Briefly, a 210 bp IRES from the human heat shock protein Bip (Banerjee et al. (1996) Transplant Proc 28:747-748) was amplified by PCR with BamHI-linked oligonucleotides from pGBip-6 plasmid (ATCC #PTA-2498) and cloned into TA cloning vector pCR2.1 (Invitrogen #K4500-01). Primers used for PCR were sense primer: taggatccgacgccggccaagaca (SEQ ID NO: 2) and antisense primer: ttaggatccagccagttgggcagc (SEQ ID NO: 3). The adenoviral gene for pIX (“pIX”) was generated by PCR amplification from the AdlinkCMVhIFNb shuttle plasmid and was cloned into TA cloning vector using sense primer: AAGGGATCCGTCGACGCCGCCATGAGC (SEQ ID NO: 4) and antisense primer: ATAGAATAGCGGCCGCAAAACCCCTAAATAAAGACA (SEQ ID NO: 5). The pIX gene was isolated from the plasmid above by Not I digest and inserted into the Not I site of the pCR2.1-Bip IRES such that the IRES is upstream of the 5′ ATG for pIX. Finally the pIX-IRES-fragment was isolated with HindIII and XhoI digestion, blunted with T4 DNA polymerase and ligated into BsaB I digested pAdlinkhCMVIFNb plasmid. The resultant plasmid was termed pCK015. In this vector the pIX gene is inverted in its natural location but the expression is directed with the IVa2 gene promoter and an IRES. This particular embodiment of such an inversion technique did not result in the generation of recombinant replication-defective adenoviruses.

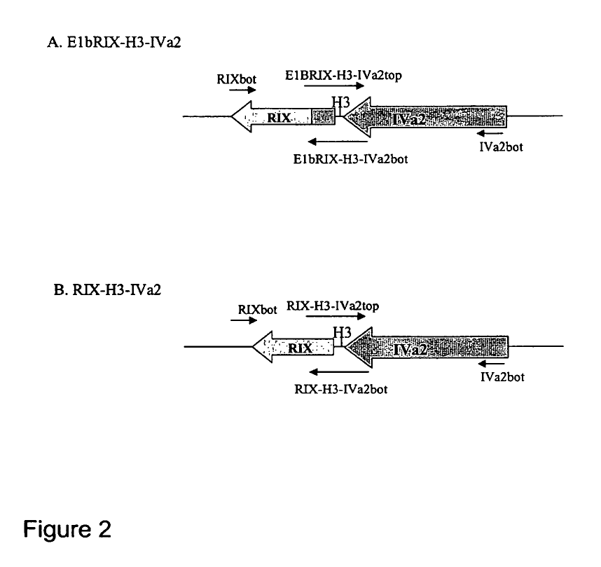

The second strategy utilized a direct inversion of the pIX gene transcription unit in its natural location. The inversion of nucleotide bases 3333-4031 in the parental vector, pAdlinkCMVIFNb, was accomplished by an overlapping PCR strategy to redesign the sequence between the unique Sal I and Bst XI sites. A unique HindIII site between the 3′ end of the IVa2 gene and the inverted pIX gene sequence was created for the insertion of a strong polyadenylation sequence to terminate the IVa2 gene transcription. The following PCR primers were employed for the overlap PCR to generate the “RIX -HindIII-IVa2” fragments: E1BRIX-H3-IVa2top 5′ tcgtacctcagcaccttcca (E1BRIX) AAGCTT gctgtctttatttaggggtt (IVa2) 3′ (SEQ ID NO: 6); RIX-H3-IVa2top 5′ acgcccacacatttc (RIX) agtacAAGCTTgctgtctttatttaggggtt (IVa2) 3′ (SEQ ID NO: 7). IVa2bot 5′ GTGATCCCAGAA ATATCTTCGCCCAG 3′ (SEQ ID NO: 8); 5′ aacccctaaataaag acagc (IVa2) AAGCTTtggaaggtgctgaggtacga (E1bRIX) 3′ (SEQ ID NO: 9); this will capture the E1b region that may enhance pIX expression; RIX-H3-IVa2bot: 5′ AACCCCTAAATAAAGACA GCAAGCTTGTACTGAAATGTGTGGGCGT 3′ (SEQ ID NO: 10), this primer only capture the minimal pIX promoter sequence; RIXbot 5′ GAATTCGTCGAC (SalI) TTAAACCGCATTGGGA GGGGAGG 3′ (SEQ ID NO: 11). See, FIG. 2. Both PCR fragments, E1bRIX-H3-IVa2 (with E1b/pIX promoter) and RIX-H3-IVa2 (pIX promoter only), were cloned back into the Sal I and BstXI site in the parental vector AdlinkCMVhIFNβ. The resultant plasmids were termed pAdlinkCMVhIFNb-E1bRIX and pAdlinkCMVhIFNb-RIX. Nucleotide sequences at the junctions were confirmed by DNA sequencing.

The human growth hormone polyadenylation signal (hGHpA) was generated by PCR amplification with the following primers: hGHpAfor—AGACAGCAAGCTTCTG CCCGGGTGG (SEQ ID NO: 12) and hGHpArev TTCCAAAGCTTCACCCCCCTCCACC (SEQ ID NO: 13). The hGHpA PCR product was flanked by HindIII sites (underlined). The product was cloned into the Hind III site between the IVa2 and the inverted pIX gene in pAdlinkCMVhIFNb-E1bRIX and pAdlinkCMVhIFNb-RIX. The resulting plasmid is termed pAdlinkCMVIFNb-E1bRIXpA and pAdlinkCMVIFNb-E1bRIXpA. It comprises (in order from the left end of adenoviral genome) the inverted terminal repeats (“ITR”), the virus packaging signal and enhancer sequences (“ψ”), the human cytomegalovirus immediate early gene enhancer and promoter (“CMV”), the SV40 human IFN-beta gene, the bi-directional SV40 polyA (“polyA”), and the pIX gene (inverted), pIX gene minimal promoter or the whole promoter/E1B enhancer region, the human growth hormone polyadenylation signal (“hGHpA”), and the protein IVa2 gene (“pIVa2”)(ATCC accession no. PTA-6198). See, FIG. 3.

### Cosmid Construction, Creation of Recombinant Adenovirus and Plaque Purification in 293 Cells

The entire IFNβ expression cassette along with the inverted pIX gene and IVa2 genes generated by the second inversion strategy described above was assembled into a cosmid for adenovirus creation. pIX modification constructs were subcloned into an intermediate cosmid pSC6 and eventually assembled into the adenovirus genome. The recombinant adenoviral genome is flanked by a unique SwaI and PacI site at the genome's 5′ end and 3′ end, respectively. The cosmid was amplified and digested with SwaI and PacI to release the genome from the cosmid backbone. The mixture of digested cosmid was transfected into 293 cells with Lipofectomine (Invitrogen) transfection reagent. After the appearance of cosmic-shaped plaques in the transfected wells, the cells were cultured and allowed to develop full cytopathic effect (in about 10 days post transfection). Cells, as well as the medium, were then harvested (crude virus). Two rounds of plaque assay were performed to purify virus clones as described previously (Graham et al. Manipulation of adenovirus vectors, Vol 7, Ed. 1 (Clifton, N.J., The Humana Press Inc.)).

### Human IFNβ Expression Analysis

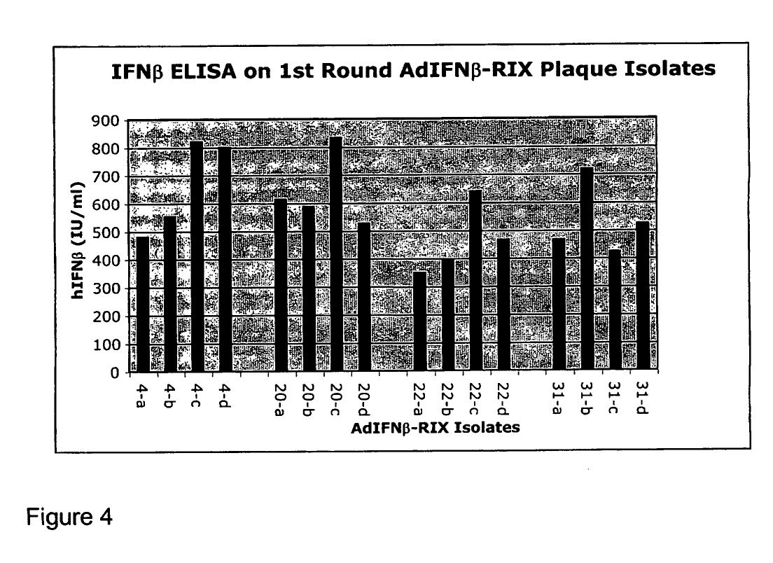

A549 human lung carcinoma cells were plated in six-well cell culture plates and infected by with 100 μl of the crude virus lysate described above. After 48 hours, the culture supernatants were assayed using an ELISA for human interferon beta (distributed by Biosource, cat. Number KAC1201). Positive controls containing dilutions of the protein were run in the presence of untransduced cell supernatants to control for media effects on detection level. FIG. 4 is a graphical representation of four clones from four inverted pIX gene (“AdIFNβ-RIX”) plaque isolates. All four clones (4a to 4d; 20a to 20d; 22a to 22d and 31a to 31d) express human IFN-β at levels that were comparable to that of the parental vector (AdIFNβ). Positive controls containing dilutions of the protein were run in the presence of untransduced cell supernatants to control for media effects on detection level.

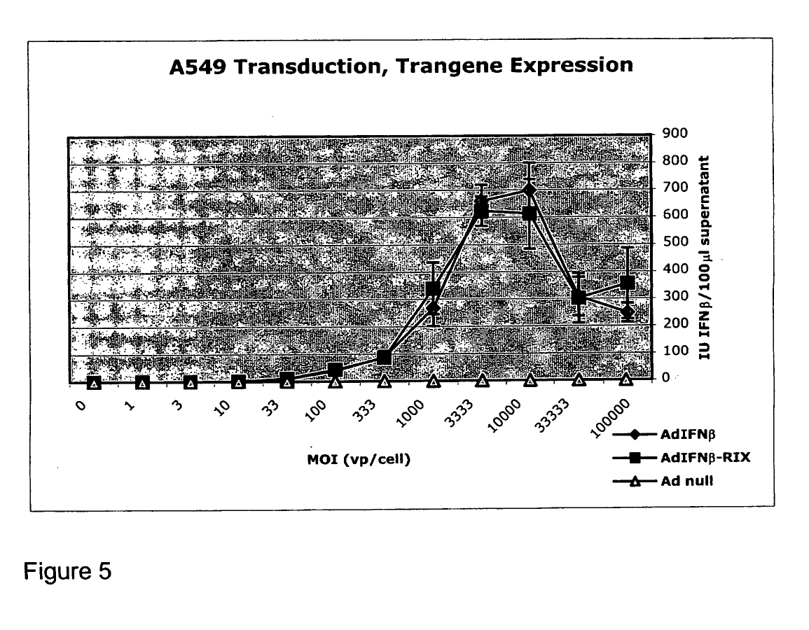

FIG. 5 is another graphical representation showing that no significant difference in IFNβ expression was detected between the AdIFNβ-RIX vector and the original AdIFNβ vector.

### High Titer Viral Stock Preparations

After two rounds of plaque purification, the viruses were grown by stepwise amplification in 293 cells, i.e., one plaque (about 4×10⁶ viral particles (“vp”)) was inoculated into a 12 well (1.5×10¹⁰ vp), then to a 10 cm dish (2.2×10¹¹ vp) and to 5×15 cm dishes (3×10¹² vp). Finally the viral lysate from the 5×15 cm dishes was used to infect a 10 layer cell factory (approx. 6320 cm², and 2.5×10¹³ vp). The virus was harvested and purified by two round of CsCl gradient centrifugation as described previously (Graham et al. Manipulation of adenovirus vectors, Vol 7, Ed. 1 (Clifton, N.J., The Humana Press Inc.)). The titer of virus stocks was determined by absorption at 260 nm. The virus yield ranges from 5×10¹² to 1×10¹³ vp.

### Viral Genomic DNA Analysis

To study the viral genomic DNA, 300 μl of the crude virus lysate were added to 293 cells in a 10 cm plate. Two days post infection, the cells were pelleted and lysed in Hirt Buffer (0.6% SDS/10 mM EDTA/100 μg Proteinase K) and the DNA was purified. Purified viral DNA was analyzed by PCR amplification for the inverted pIX gene and restriction analysis for genome structure.

### Serial Passage Test for RCA Generation

Two strategies of serial passaging were employed to generate RCA. One strategy employed small scale serial passaging in which 293 cells (5×10⁶) were cultured in 150 mm dishes. Once cell density reached about 80%, the cells were infected with the parental IFNβ vector (AdIFNβ), the control vector (AdIFNβ-M9)_ or the inverted pIX (AdIFNβ-RIX) vector MSS-1 virus stocks at a multiplicity of infection (MOI) of 200. Each passage of virus was termed as master seed stock or MSS. The number behind the MSS indicates the passage number. The assay was carried out in triplicates. When CPE fully developed (usually takes about 3 days), both cells and the medium were harvested and prepared into crude viral lysates (“CVL”) by three rounds of freeze and thaw. Approximately 1/100 (usually 0.2-0.5 ml) of the CVLs was then used to infect new 293 cells in 150 mm dishes and this procedure was repeated to create a series of crude virus lysates numbered P1, P2, P3 and so on. This assay for each vector was carried forward until the detection of RCA. All CVLs were stored at −80° C. for RCA analyses.

The second strategy used to evaluate the possibility of RCA emergence at later passages of viral replication involved large scale serial passaging in which 293 cells (6×10⁸) were seeded in a 10 layer cell factory (Corning) and cultured for 2 days before infection with vector seed stocks. The cells were infected with MSS-1 of AdIFNβ, AdIFNβ-M9 or AdIFNβ-RIX at a MOI of 200 (which is approximately ˜1×10¹¹ viral particles per cell factory). At 48 hours post infection, cells were harvested and viruses were purified by two rounds of CsCl gradient purification. The new viral stocks were termed as MSS-2 and will be used to infect new 293 cells (6×10⁸) for the next round of passage to generate MSS-3, MSS-4 and so on. The advantage of this assay is to allow enough accumulation of amplified viral particles for viral DNA isolation. The isolated viral DNA can be purified further to remove 293 cellular DNA contamination before RCA analysis. The passages were stopped when RCA became detectable.

To detect RCA contamination in either the high titer viral stocks or the CVLs generated from small-scale serial passage test, quantitative RCA (QPCR) and liquid culture assay were used. Q-PCR procedure is dependent on purified viral DNA, so that it is mainly used in analyzing the high titer viral stocks. In contrast, liquid culture assay solely depends on live virus and is used for confirmation of the QPCR results as well as for detection of RCA in CVLs.

### RCA Detection

Real-time PCR for the presence of E1 sequence was performed. Three sets of primers and probes were designed for this assay. The first set was used to detect the adenovirus E1 region for RCA, the second set was used to detect the E2a gene to quantify the total viral particle amount, and the third set was used to detect the 18S ribosomal DNA for monitoring the levels of 293 cellular DNA contamination. The primer sequences are listed in Table 1 below.

TABLE 1

QPCR primers and probes for RCA detection

| | | |
|---|---|---|
| Primer | Sequence | Homology Region |

| | | |
|---|---|---|
| E1 | AGATACACCCGGTGGTCCC | matches nucleo- |
| forward | (SEQ ID NO: 14) | tides 1400-1418 of |
| primer | | wildtype Ad5 se- |
| | | quence (GenBank # |
| | | M73260) |
| E1 | CGACGCCCACCAACTCTC | matches nucleo- |
| reverse | (SEQ ID NO: 15) | tides 1463-1446 of |
| primer | | wildtype Ad5 se- |
| | | quence |
| E1 | CTGTGCCCCATTAAACCAGTTGC | matches nucleo- |
| probe | CG | tides 1420-1444 of |
| | (SEQ ID NO: 16) | wildtype Ad5 se- |
| | | quence |
| E2a | TTGCGTCGGTGTTGGAGAT | matches nucleo- |
| forward | (SEQ ID NO: 17) | tides 23024-23042 |
| primer | | of wildtype Ad5 |
| | | sequence |
| E2a | CAAGGCCAAGATCGTGAAGAA | matches nucleo- |
| reverse | (SEQ ID NO: 18) | tides 23088-23068 |
| primer | | of wildtype Ad5 |
| | | sequence |
| E2a | TGCACCACATTTCGGCCCCA | matches nucleo- |
| probe | (SEQ ID NO: 19) | tides 23044-23063 |
| | | of wildtype Ad5 |
| | | sequence |
| 18S | GCCTGCGGCTTAATTTGACT | matches nucleo- |
| forward | (SEQ ID NO: 20) | tides 1229-1248 of |
| primer | | human 18S rRNA se- |
| | | quence (GenBank |
| | | # X03205) |
| 18S | CAACTAAGAACGGCCATGCA | matches nucleo- |
| reverse | (SEQ ID NO: 21) | tides 1348-1329 of |
| primer | | human 18S rRNA se- |
| | | quence |
| 18S | AAAGAGCTATCAATCTGTCAATC | |
| probe | CTGTCCGTGT | |
| | (SEQ ID NO: 22) | |

In order to extract the viral DNA from adenovirus virions for PCR amplification, 2×10¹² Vp of master seed stocks of AdIFNβ, AdIFNβ-M9 or AdIFNβ-RIX was treated with benzonase to remove cellular DNA contamination since 293 cell DNA contains E1 sequences. The digestion was setup in 20 mM Tris-HCl, pH8, 2 mM MgCl₂, 20 mM NaCl buffer, 10 ul Benzonase (about 2000 units) and incubated at 37° C. for 2 hours. The reaction was mixed with 2× CsCl solution (1.024 g CsCl/ml) for gradient centrifugation to purify the viral particle. This step is necessary to separate the viral particle from benzonase digestion-generated DNA fragments. At the end of centrifugation, viral bands were harvested and dialyzed against PBS solution. Purified viral particles were then treated with protease K (100 ug/ml) at 37° C. for 2 hours, extracted with phenol/chloroform and precipitated by ethanol. Finally DNA pellets were dissolved in TE buffer and stored at 4° C. for QPCR analyses.

The viral genome copy numbers were normalized using the linearized adenovirus plasmid, pFG140 as the E1 and E2a standards. The levels of 293 cell DNA was used as the standard for 18S determination. The RCA levels were calculated as: (E1 #-18S#)/E2a×10¹⁰—i.e., RCA per 1×10¹⁰ vp.

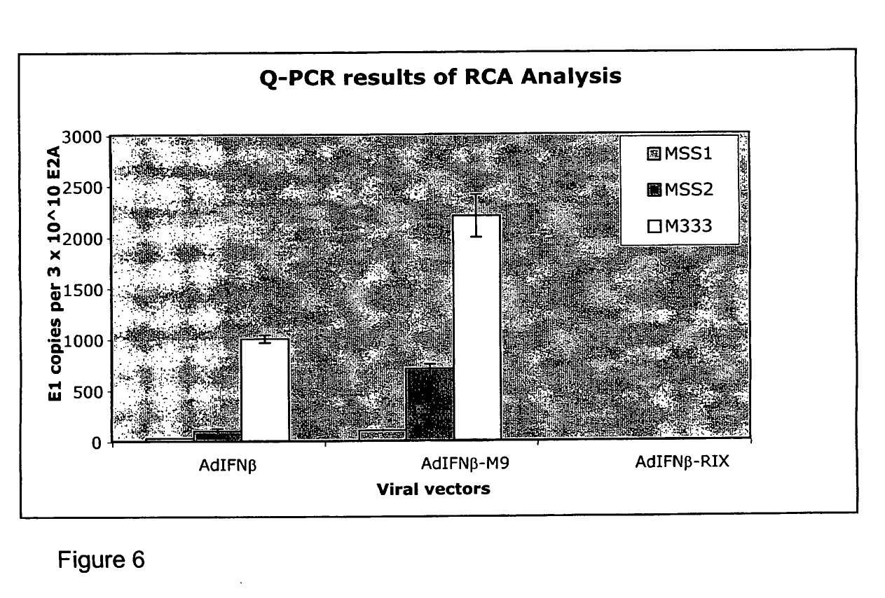

FIG. 6 is a graphical representation of the real time PCR (Q-PCR) results of E1 detection in serial passages of viral stocks. Parental IFNβ vector (AdIFNβ), a control vector (AdIFNβ-M9, which is derived from the parental IFNβ vector containing mutations in the pIX gene), and three of the inverted pIX (AdIFNβ-RIX) vectors were serially passed three times (Master Seed Stock (MSS)—MSS1, MSS2 and MSS3) in 293 cells. The viral DNA was isolated and E1 gene copies were determined using Q-PCR. RCA was detectable in AdIFNβ MSS3 and in AdIFNβ-M9 MSS2 and MSS3. In contrast, no RCA was detectable in all three MSS passages in AdIFNβ-RIX vector. These results suggest that the mutation of the pIX gene in the AdIFNβ-M9 control vector may not be sufficient to suppress the emergence of RCA. Therefore, the large scale passage for AdIFNβ-M9 vector and AdIFNβ control was stopped at MSS4 whereas the AdIFNβ-RIX vector passage was continued until passage 10. The AdIFNβ MSS1-MSS4 and AdIFNβ-RIX MSS1-MSS10 passages were purified by benzonase treatment and CsCl purification and viral DNA samples were isolated and analyzed again by Q-PCR.

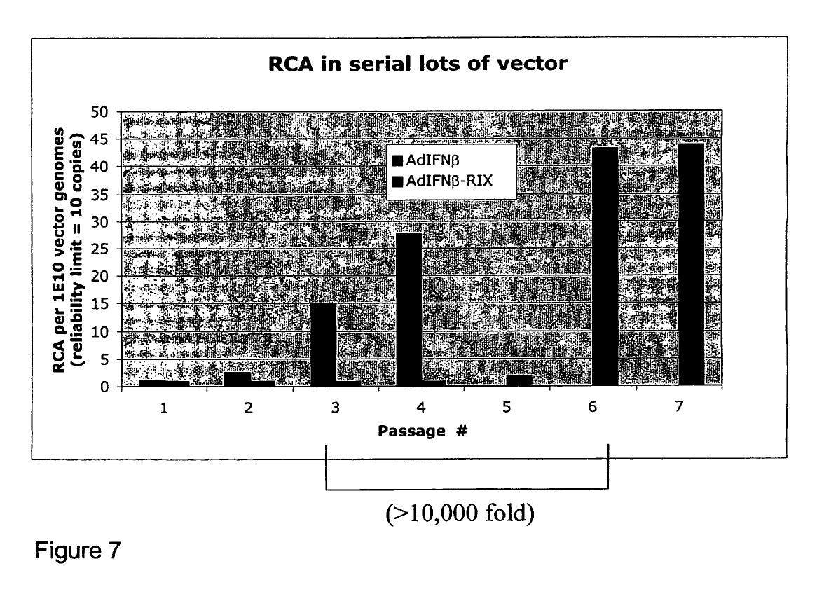

FIG. 7 is a graphical representation of the RCA emergence in serial passages of the AdIFNβ and AdIFNβ-RIX vectors. The results clearly demonstrate that the emergence of RCA was delayed in the AdIFNβ-RIX vector for three passages as compared to the AdIFNβ vector. The folds of reduction were calculated based on the virus yield for each run and inoculation ratio in each passage. The results demonstrate that a 10,000 fold reduction in RCA incidence in the AdIFNβ-RIX vector as compared to the AdIFNβ vector.

Although the Q-PCR assay results clearly demonstrated the significant delay of RCA emergence and the decrease E1 gene copy numbers in viral DNA preparations, these results do not represent true active viruses. Even though cellular DNA contamination can be ruled out by the 18S rRNA control, the presence of E1 positive, atypical RCA cannot be distinguished from typical RCA using PCR based methods. The real-time PCR results were thus further confirmed using the liquid culture method, which is a more reliable assay in the determination of infectious units.

The liquid culture assay is a three-step “absorption-amplification” procedure to maximize the sensitivity of the assay. To further reduce the toxic effect of human IFNβ, an IFNβ antibody (BO2) was added into virus infected cells at 1 μg/ml concentration to block IFNβ function. The assay involves absorption of the vector into HeLa cells, transfer of putative RCA to A549 cells followed by 28-30 day culture to amplify replicative recombinants. The third step is to assay culture supernatants from the A549 cells on naive A549 cells to verify the presence of replicative virions. The rationale for this approach is that HeLa cells can withstand more adenovirus vector toxicity (Hehir et al., J Virol 70:8459-8467 (1996)) and are less susceptible to the deleterious effects of the interferon transgene present in the adenoviral vector of this invention than A549 cells, but they are not as proficient at replicating RCA as the A549 cells. The non-replicative vector is transiently expressed from the HeLa target cells but does not proliferate and primarily the replicating recombinants (RCA) are then passed on to the A549 cells. Even with the absorption step, the A549 cells often respond to the presence of vector or the transgene in a way that mimics CPE. However, “real” RCA is easily distinguished from these “pseudo-CPE” effects by the ability to transduce and proliferate on naive cells.

Briefly, HeLa cells plated at a concentration of 1.5×10⁷ cells in 3×150 mm plates (80% confluent) were transduced with 6×10¹⁰ viral particles of either the purified cell factory produced material or the serial-passaged crude viral lysate (“CVL”). Positive controls containing early passage CVL (with no evidence of RCA) were spiked with 0.5 pfu, 5 pfu, 50 pfu, or 500 pfu of the wild-type Adenovirus 5.

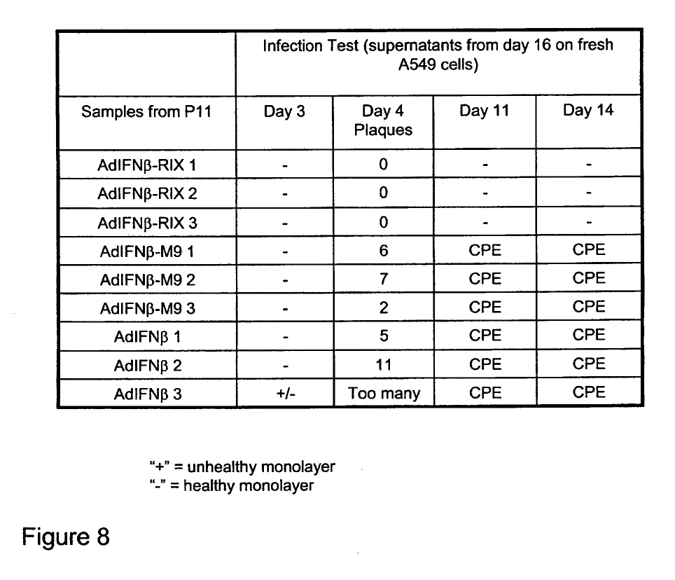

FIG. 8 is a table summarizing the liquid culture results. The same lots of viruses used in the Q-PCR assay shown in FIG. 6 were analyzed by liquid culture method for the presence of RCA. “+” symbols indicate the levels of RCA detected. Parental vector (AdIFNβ 1-3), inverted pIX vector (AdIFNβ-RIX1-3) and control vector (AdIFNβ-M91-3) were serially passaged in 293 cells up to passage 11 (P11) and then subjected first to absorption on HeLa cells. Next, any putative RCA would be transferred to A549 cells followed by culturing to amplify replicative recombinants. Finally, culture supernatants were assayed on naive A549 cells to verify presence of replicative virions and the results are displayed in the table as “Infection Test (supernatants from day 16 on fresh A549)”. No RCA was detected in all three rIX stocks by this method and these results confirmed the above Q-PCR data. This is indicated in the last column by the lack of cytopathic effect (“CPE”).

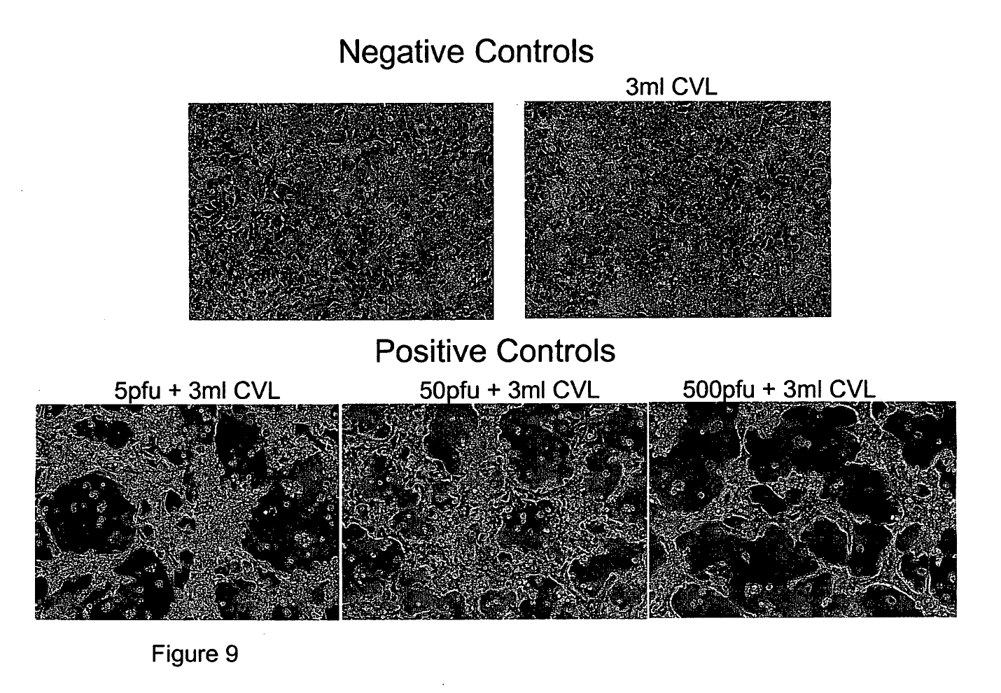

FIG. 9 are photomicrographs from the liquid culture assay displaying the cell morphology at day 7 after infection of HeLa cells with the wild type adenovirus 5 (“Ad5”) in the presence of AdIFNβ vector (to control the IFNβ effects on plaquing efficiency). The top panels display the cell morphology of negative controls in which either media control or 3 mL of crude viral lysates (“CVL”) containing an adenoviral vector of this invention was added. The positive controls, shown in the bottom panels, display the morphology of cells containing early passage CVL (with no evidence of RCA) and spiked with either 5 pfu, 50 pfu or 500 pfu of the wildtype Ad5. As low as 5 pfu can be detected in this assay in the presence of IFNβ expression. These results indicate that cells with CVL containing the inverted pIX adenoviral vector displayed no cytopathic effects as compared with the cells in the positive controls.

After four days of culture, the cells were harvested with the media, pelleted, and the pellets subjected to three cycles of freeze (liquid nitrogen) and thaw (37° C.). The disrupted cell pellets were suspended with 2.5 ml of the original culture supernatant, the debris was pelleted and the RCA-containing supernatant applied to 1.5×10⁷ A549 cells in a 15 cm plate. Cells were cultured for 10 to 15 days with periodic monitoring of the health of the cells and observation for signs of CPE. When the monolayers became unhealthy due to over-confluency, the cells were harvested, pelleted and replated onto fresh 15 cm culture plates. The cultures were continued for another 10 to 15 days. At any point during the liquid culture and always at the end of the culture period, if the visual observations were unclear, a 100 μl sample of the culture supernatant was assayed on naive A549 cells in a 6-well plate to monitor for evidence of replicating RCA.

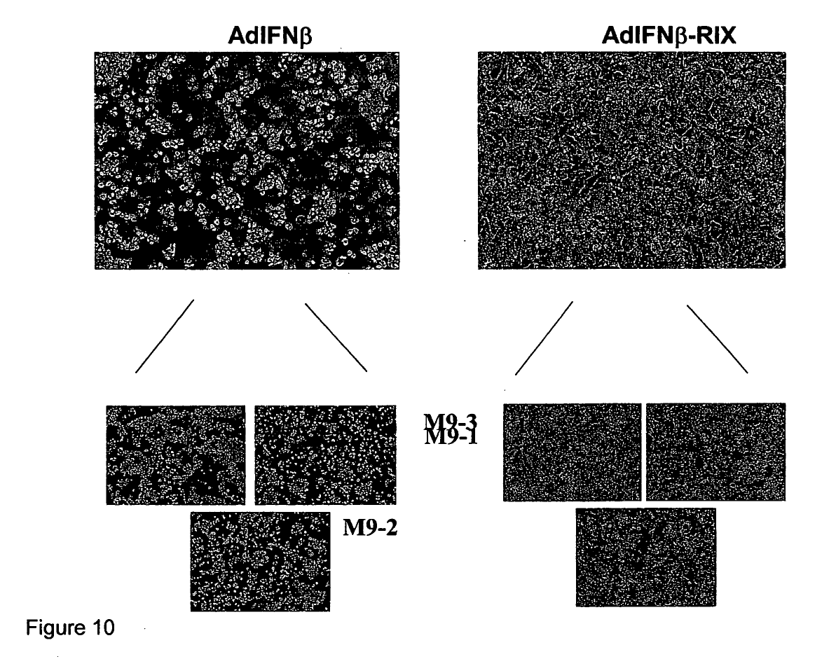

FIG. 10 is the liquid culture results of inverted pIX (AdIFNβ-RIX) vectors and parental vector (AdIFNβ)). The photomicrographs in the top panel pictures shows the A549 cell infection results, indicating the absence of RCA in RIX stocks (MSS3). The photomicrographs in the lower panel show the confirmation result of infection of fresh naive A549 cells using the lysate from the original A549 test. This further confirms the absence of RCA in RIX containing cells and ruled out the possibility of IFN-β induced cell death effects.

In another set of experiment, four days after culturing in the presence of BO2, cells were harvested and lysed in 2.5 ml fresh medium by 3× freeze and thaw cycles. Cell debris was cleared by centrifugation and the entire supernatant was used to infect a 150 mm dish that contained about 80% confluent A549 cells. The cultures were maintained for an additional 20 days for RCA plaque appearance and CPE counting. As shown in FIG. 11, the emergence of RCA was delayed three passages in the AdIFNβ-RIX vector, thus confirming the Q-PCR results described in FIG. 7.

Small scale liquid culture passage experiment also demonstrated the reduced incidence of RCA in AdIFNβ-RIX vector. As an independent experiment, AdIFNβ and AdIFNβ-RIX were tested in a serial passage experiment in 293 cells. Briefly, 3×10⁸ viral particles from MSS1 of AdIFNβ-RIX and AdIFNβ were used to infect 293 cells in 150 mm culture dishes (MOI about 60). The experiment was carried out in triplicates. Based on the QPCR results discussed above, the contamination of RCA in the starting inoculation is close to zero. Therefore, all detected RCA in the serial passage presumably are from novel recombination events between the vectors and the 293 cells DNA. After cell lyses reached completion (usually 5 days), cell debris and medium were harvested and underwent 3 rounds of freeze/thaw cycle to release all viral particles. Approximately 1/100 (˜200 μl) of the crude lysate were used for the next round of passage until detection of RCA.

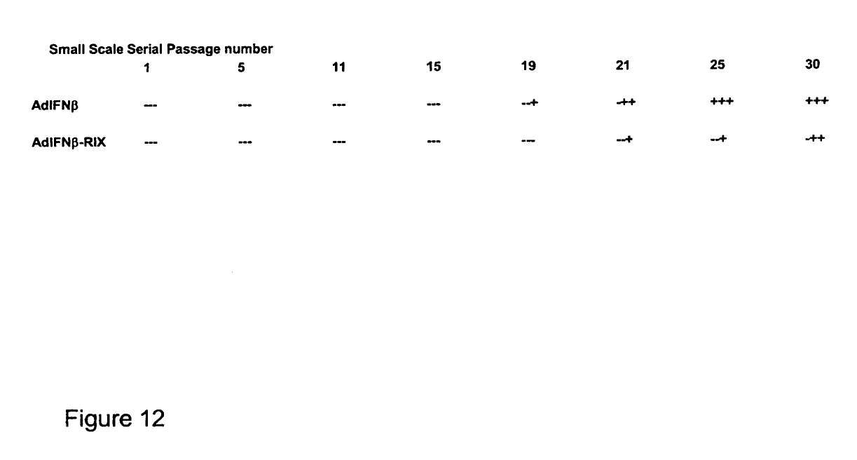

As shown in FIG. 12, small scale serial testing for RCA resulted in reduced incidence of RCA in AdIFNβ-RIX vector. RCA were detected at passage 19 in ⅓ AdIFNβ. In contrast, only one AdIFNβ-RIX plates showed RCA at passage 21, the second plate turned to RCA positive at passage 25 and the third one remained RCA free through passage 30 (experiment termination point).

### Thermostablility

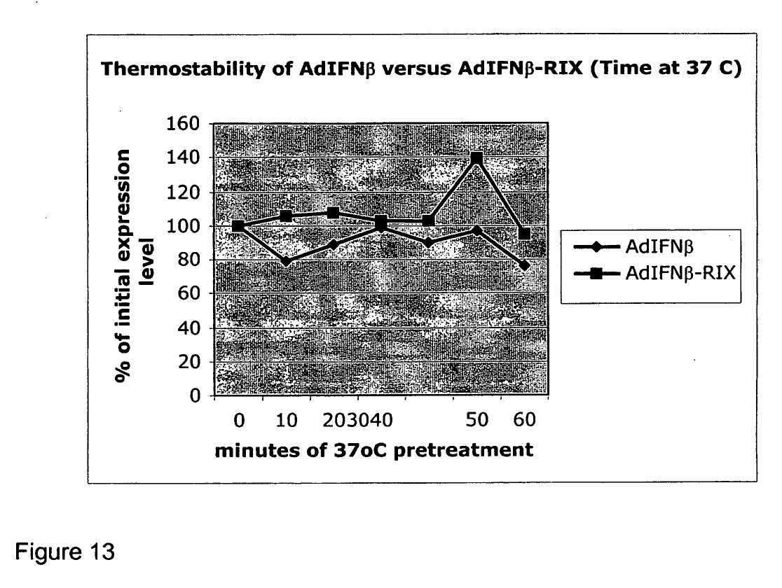

Adenovirus pIX has both a structural and regulatory function for virus assembly and replication. Therefore, pIX gene manipulation in the viral backbone can potentially change the levels of pIX protein and affect its functions. Since one of the critical functions of pIX on viral capsid is to provide for viral thermostability (Colby and Shenk, J Virol 39:977-980 (1981)), the effect of inverting the pIX gene in the AdIFNβ-RIX vector was studied. The thermostability of the AdIFNβ-RIX vector was tested by function of incubation time at 37° C. Briefly, AdIFNβ-RIX and AdIFNβ aliquots containing 1×10¹¹ viral particles were made and incubated at 37° C. for 10-60 minutes. The viral particle infectivity was assessed by infecting A549 cells in 12 well plates in triplicates at MOI of 1,000, at which levels of hIFNβ expression reaches about 50% of maximal hIFNβ expression under same culturing conditions, as shown in FIG. 5, and represents the most sensitive stage for viral titer change. IFNβ production was used as indirect measurement of activity of the viral particles. As shown in FIG. 13, no significant difference in thermostability was found between the AdIFNβ-RIX and AdIFNβ vector at any incubation time.

The thermostability of the AdIFNβ-RIX vector was next tested under increasing temperature conditions. AdIFNβ-RIX and AdIFNβ aliquots containing 1×10¹¹ viral particles were made similarly and incubated in an increasing temperature gradient (37-56° C.) on a PCR machine for 60 min (DNA Engine PTC-200, MJ Research). The virus samples were then used to infect A549 cells in 12 well plates in triplicates at MOI of 1,000. The level of IFNβ production was used as indirect measurement of activity of the viral particles.

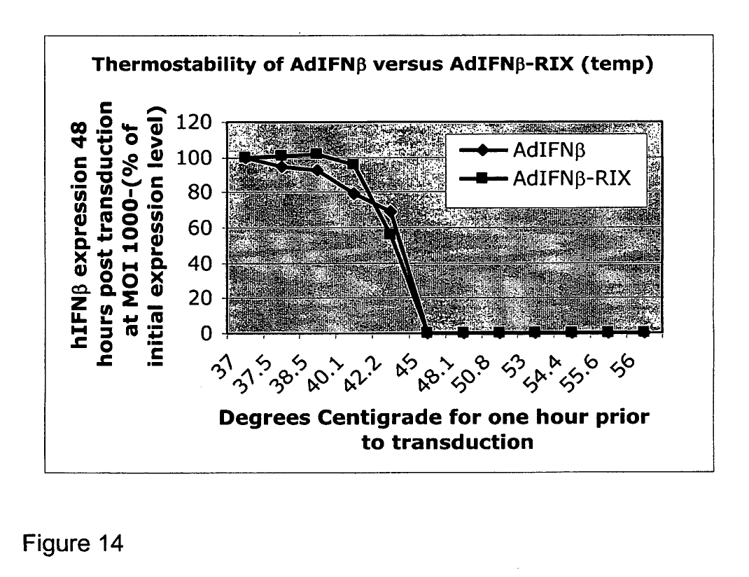

As shown in FIG. 14, no statistical difference was found between AdIFNβ-RIX and AdIFNβ in their thermostability profiles. Both IT₅₀ (inactivation of 50% of the viral activity) was observed around 43° C., and 100% inactivation was observed at 45° C.

### HPLC and AUC Analyses

Finally, the integrity and purity of AdIFNβ-RIX virus preparation was characterized by HPLC and analytical ultra-centrifugation (AUC) analyses. Both methods were able to detect empty capsid, free viral proteins released during preparation process and virus aggregates.

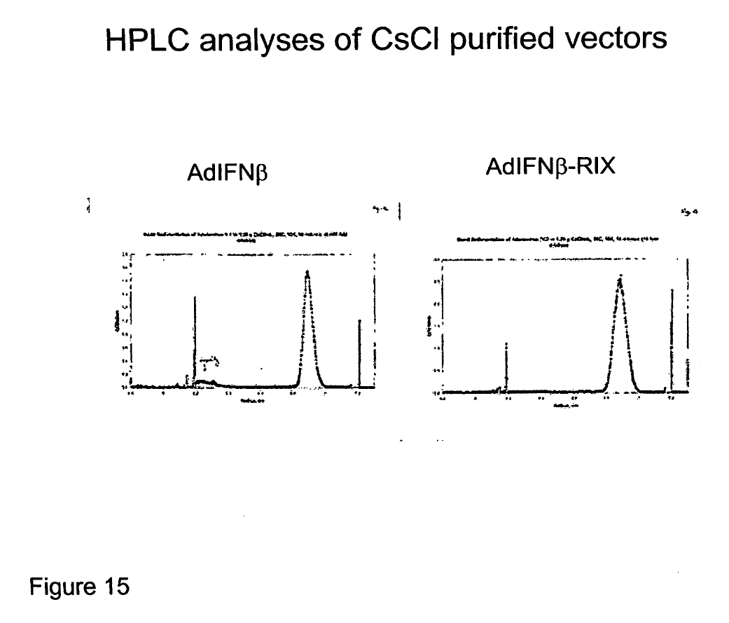

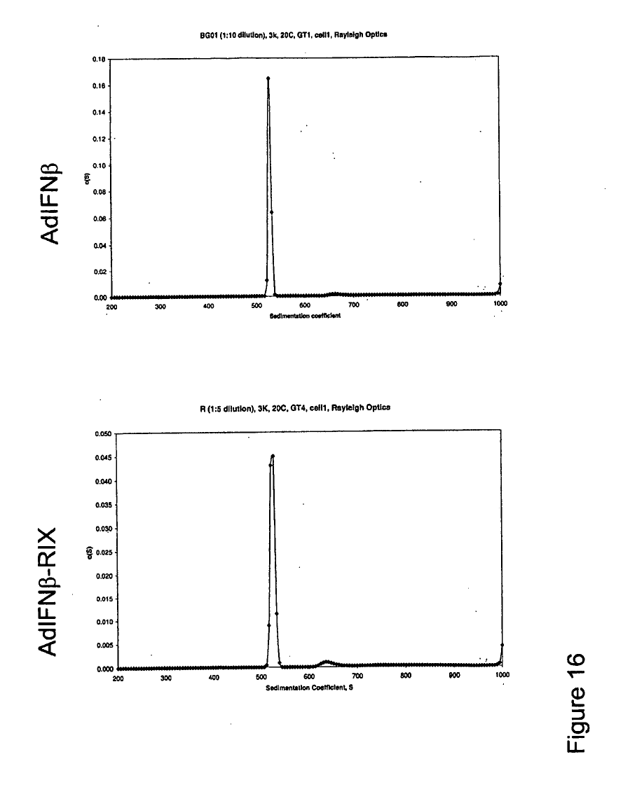

FIG. 15 is the viral particle analysis by HPLC. The panels include viral particles containing the parental vector (AdIFNβ), and the inverted pIX gene (AdIFNβ-RIX). The results indicate that the viral particles are indistinguishable from the parental vector (AdIFNβ). Similarly, when AdIFNβ-RIX vectors were analyzed by AUC which captures empty capsid, free proteins and aggregates, the histogram of AdIFNβ-RIX is very similar to AdIFNβ (as shown in FIG. 16. These results suggested that the pIX gene manipulation does not change the virus characteristics.

### EXAMPLE II

Effect of the Inverted PIX-Containing Adenoviral Vector in an in vivo Xengraft Tumor Model

The effect of the inverted pIX-containing adenoviral vector in in vivo models was studied in the xenograft tumor model in nude mice.

Briefly, 1×10⁶ U87 cells, a human glioma cell line, were injected subcutaneously into nude mice to establish tumor in the right lateral dorsal flank area. One week following cell implantation, tumors were treated with the AdIFNβ-RIX vector (at 1×10¹¹ viral particles/tumor), parental vector (AdIFNβ) or control vector (AdLacZ). Tumor size was monitored and measured using calipers. The tumor volume was calculated as LW²/2, where L represents the length of the tumor and W represents the width of the tumor. The experiments were terminated three weeks post treatment due to severe tumor ulceration of the AdLacZ control group animals.

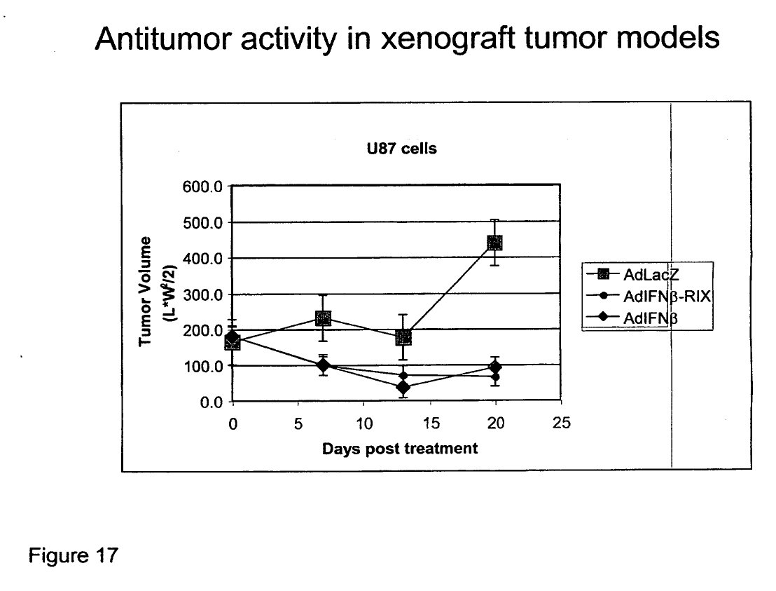

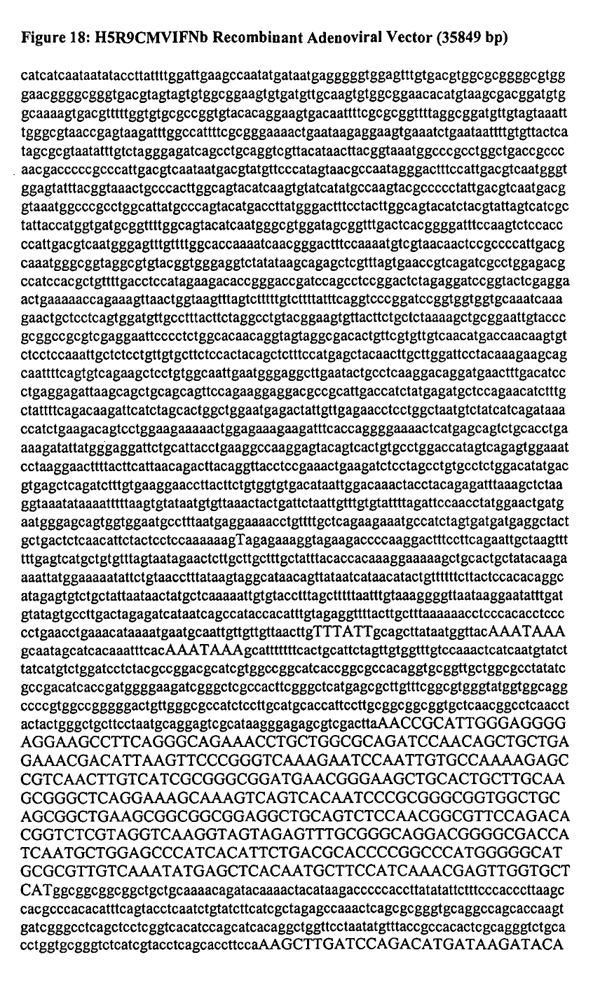

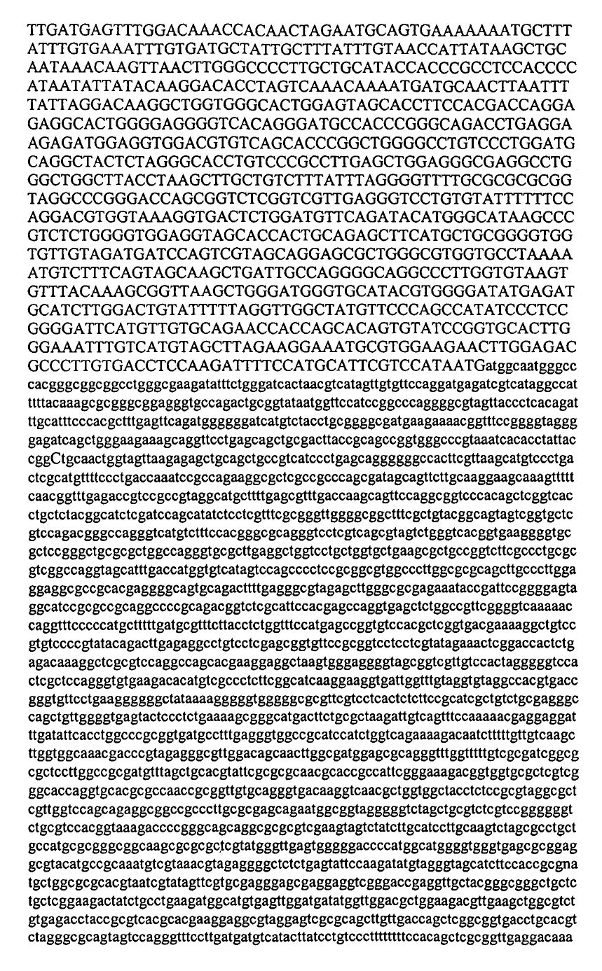

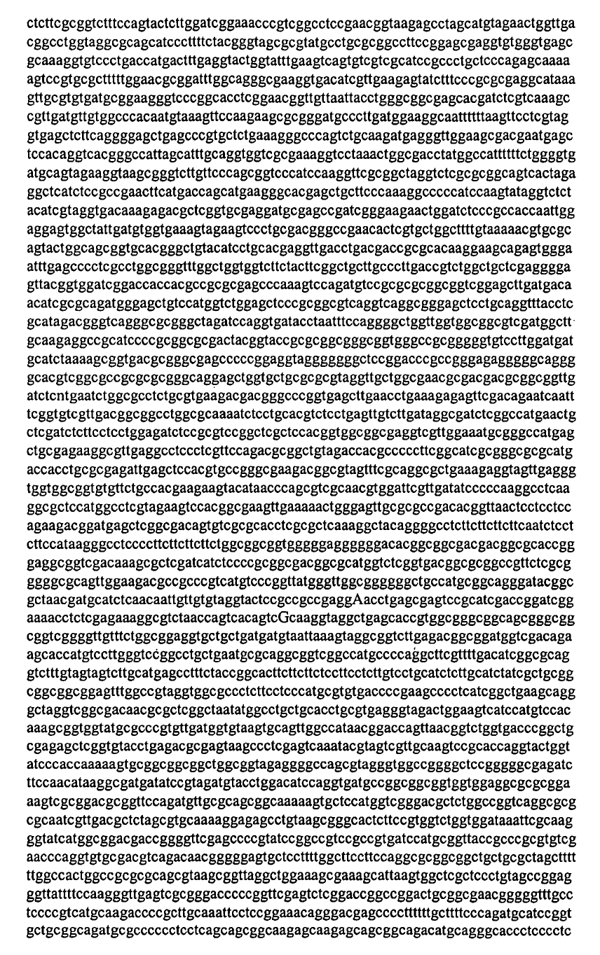

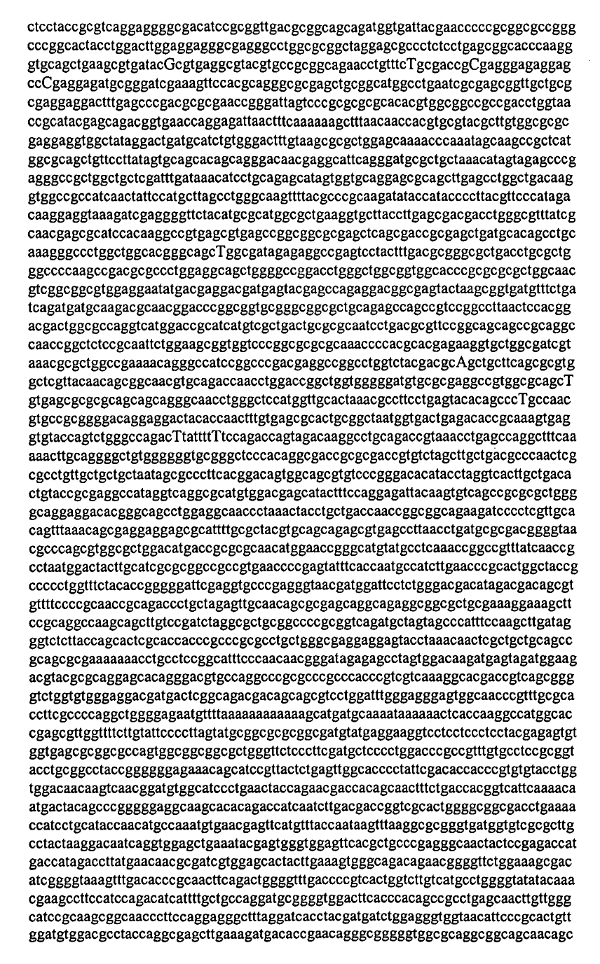

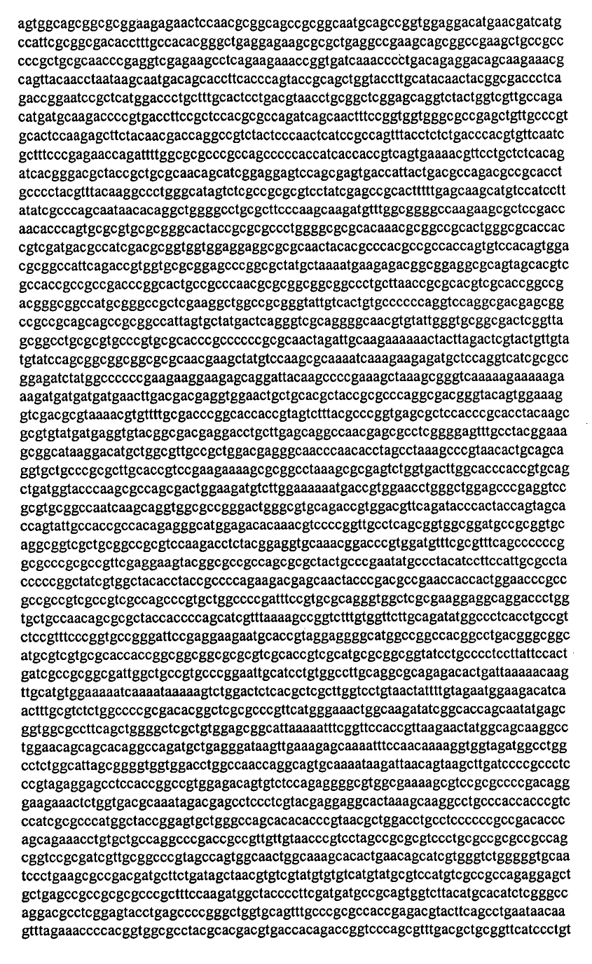

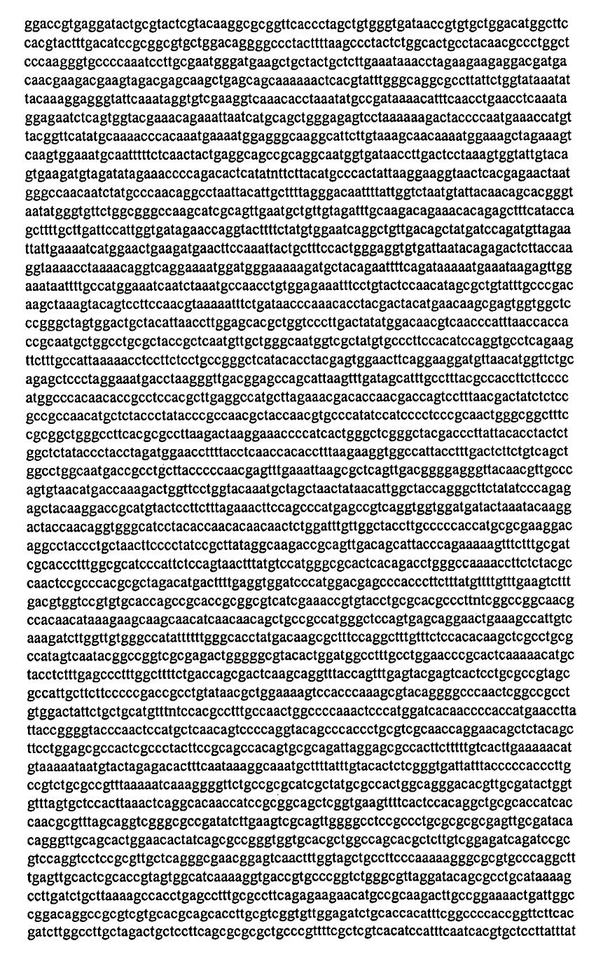

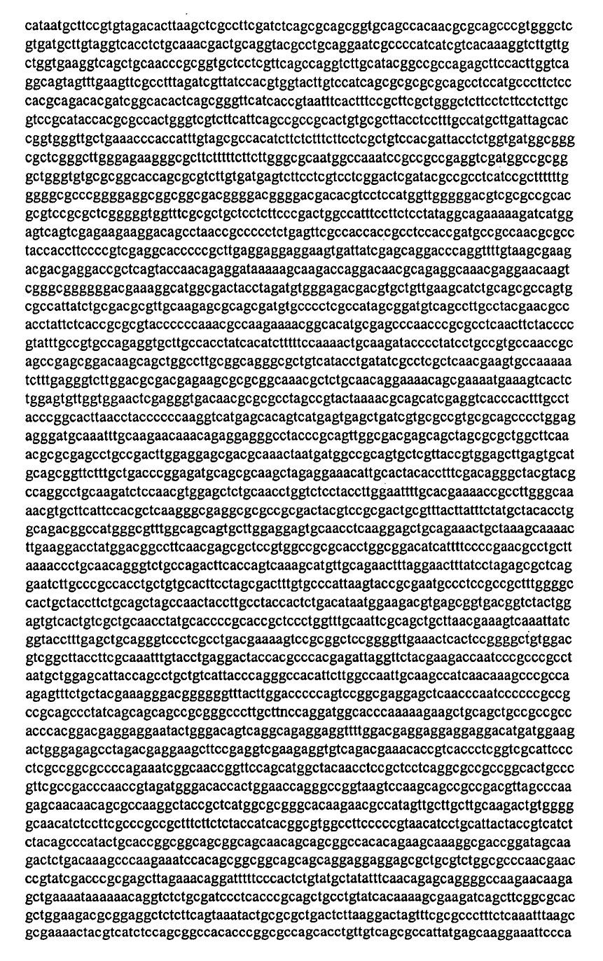

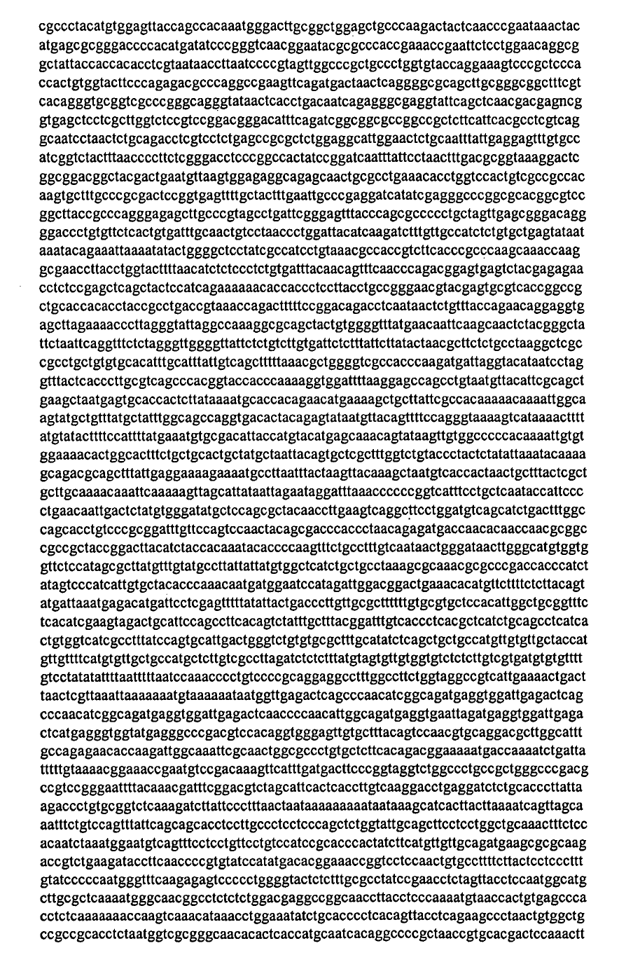

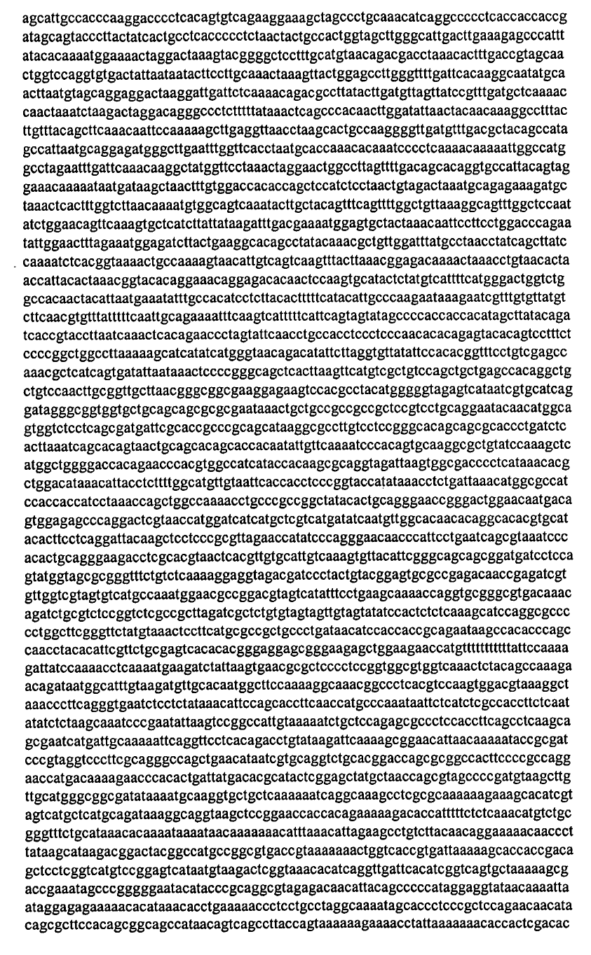

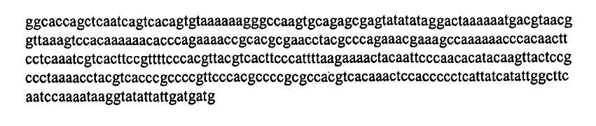

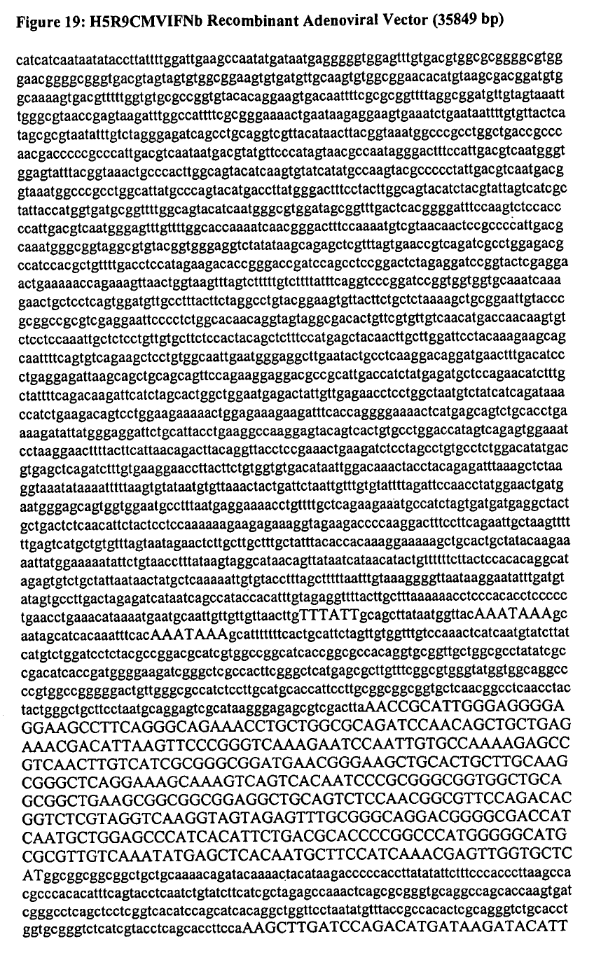

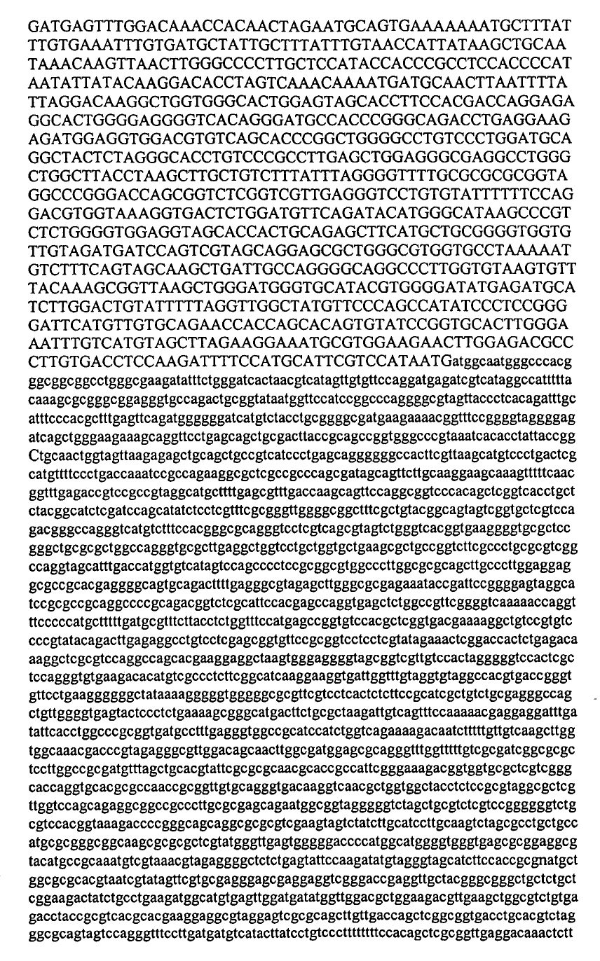

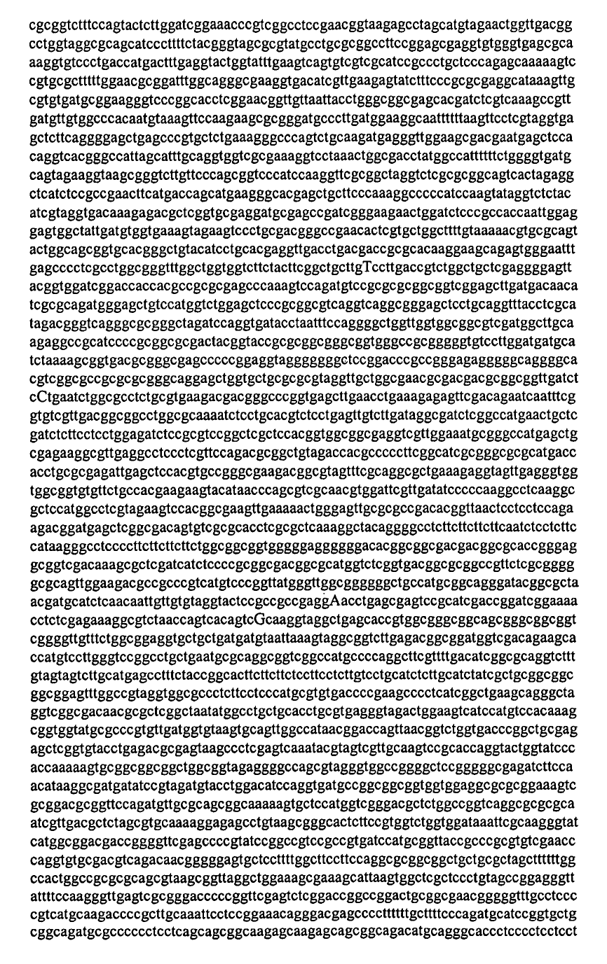

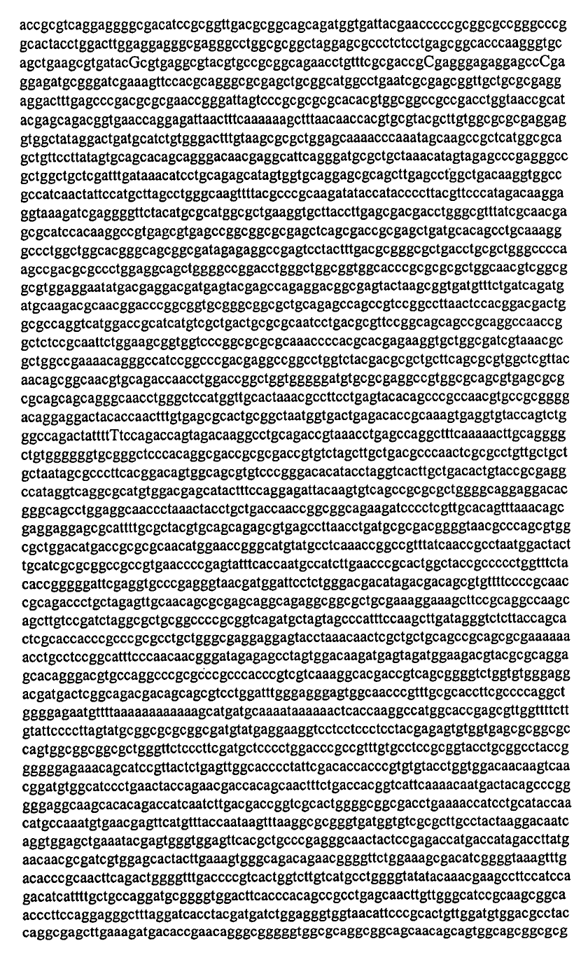

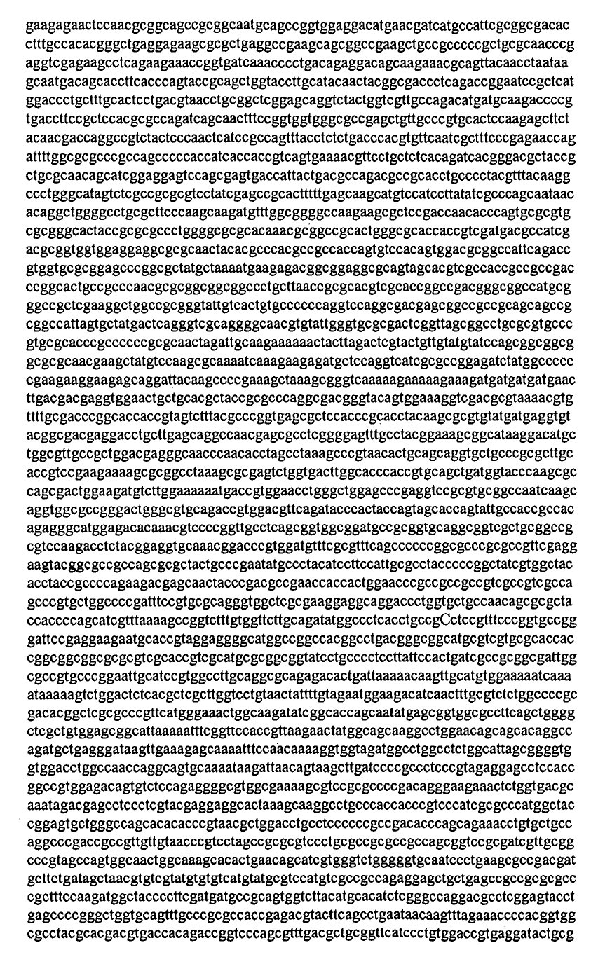

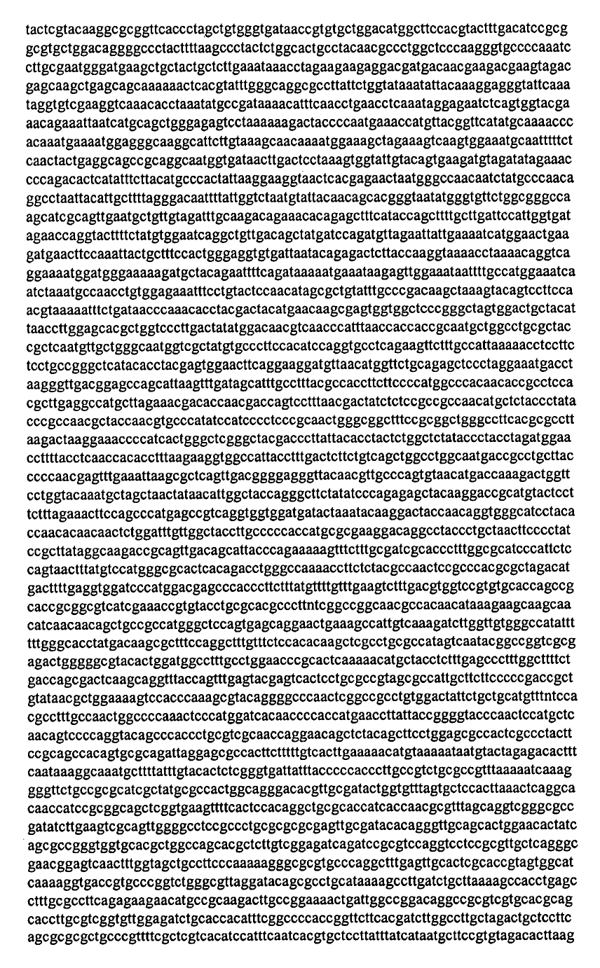

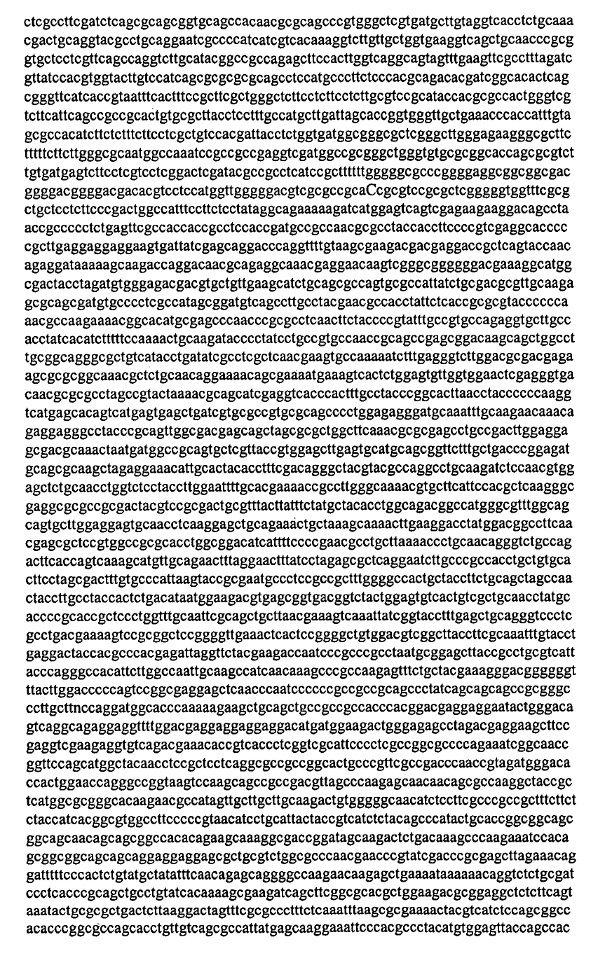

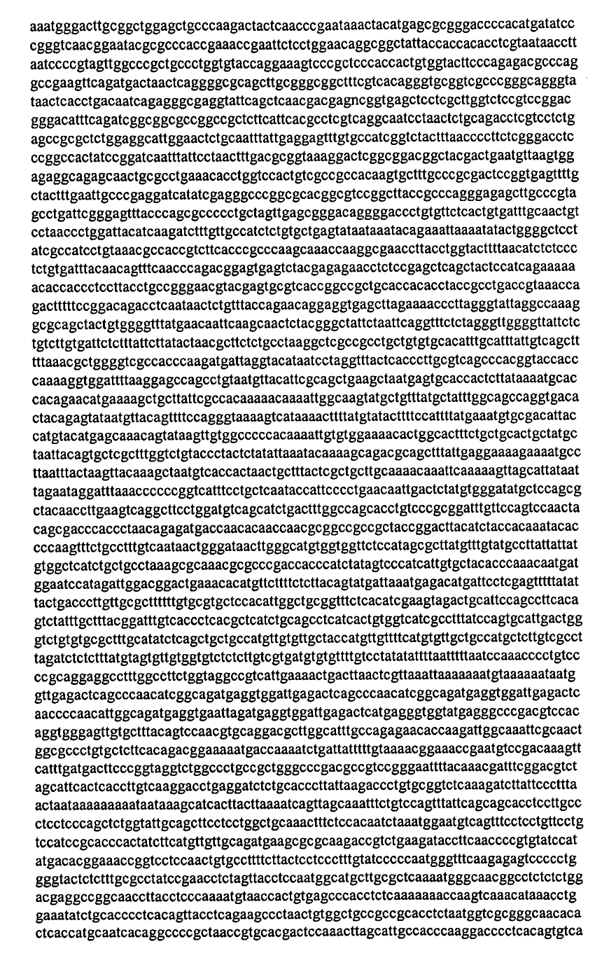

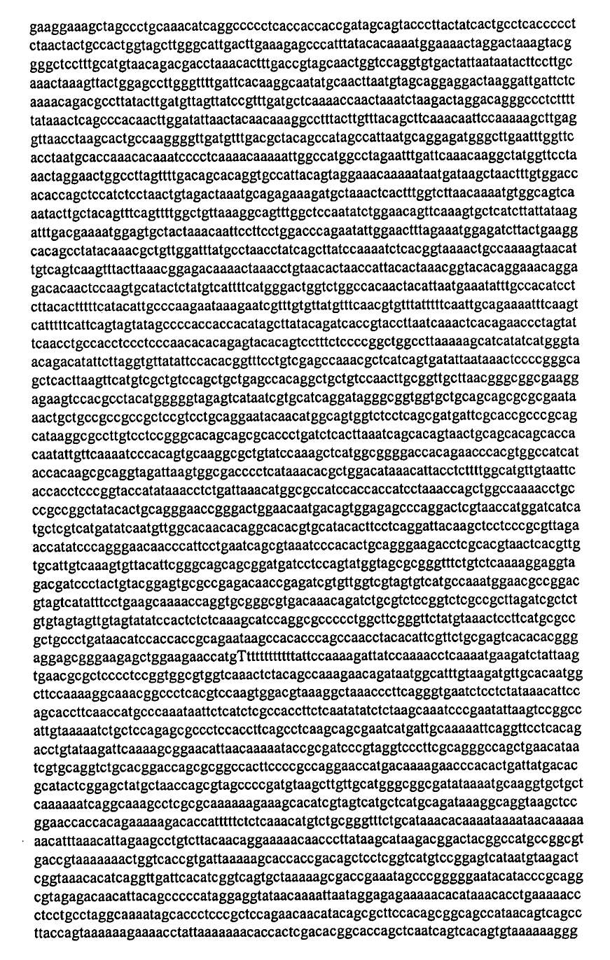

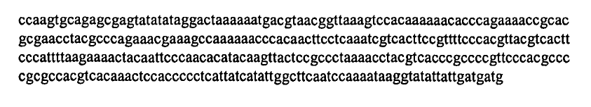

FIG. 17 illustrates that both the RIX and parental vectors showed comparable tumor killing activities, indicating that modification of the pIX gene did not alter either the IFN-β expression levels or the AdIFNβ tumor killing capability.

1. A recombinant replication-defective adenoviral vector comprising:
(a) an adenoviral genome in which at least a part of the E1 region of the adenoviral genome is deleted; and
(b) wherein on said adenoviral genome the protein IX gene is in an inverted orientation relative to the direction of transcription of the native protein IX gene at a location where the protein IX gene normally resides, wherein said inverted protein IX gene is operably linked to regulatory elements thereby allowing its expression in a host cell.

2. The adenoviral vector according to claim 1, wherein said recombinant adenoviral vector comprises a gene of interest, and which said gene of interest is operably linked to regulatory elements thereby allowing its expression in a host cell.

3. The adenoviral vector according to claim 1, wherein all of the E1 region of the adenoviral genome is deleted.

4. The adenoviral vector according to claim 1, wherein said inverted protein IX gene is placed under the control of the protein IX natural promoter.

5. The adenoviral vector according to claim 2, wherein said gene of interest is placed under the control of the cytomegalovirus immediate early promoter (CMV promoter).

6. The adenoviral vector according to claim 2, wherein said gene of interest encodes a protein selected from the group consisting of LIGHT, interferon-β, herpes simplex virus thymidine kinase and p53.

7. The adenoviral vector according to claim 6, wherein said gene of interest encodes LIGHT.

8. The adenoviral vector according to claim 6, wherein said gene of interest encodes interferon-β.

9. The adenoviral vector according to claim 8, wherein said interferon-β is human interferon-β.

10. A viral particle comprising the adenoviral vector according to claim 1.

11. The viral particle according to claim 10, wherein said particle is made in a human embryonic kidney 293 cell.

12. An isolated host cell comprising the adenoviral vector according to claim 1.

13. The host cell according to claim 12, wherein said host cell is a mammalian cell.

14. The host cell according to claim 13, wherein said mammalian cell is a human cell.

15. The host cell according to claim 14, wherein said human cell is a human embryonic kidney 293 cell.

16. The host cell according to claim 12, wherein there are insufficient overlapping sequences between said adenoviral genome and said host cellular genome to appreciably mediate a recombination event sufficient to result in a replication-competent adenovirus.

17. The host cell according to claim 16, wherein said overlapping sequences in the right-arm region between said adenoviral genome and said host cellular genome are no more than 280 bp.

18. The host cell according to claim 17, wherein said overlapping sequences in the right-arm region between said adenoviral genome and said host cellular genome are no more than 264 bp.

19. A preparation that is substantially free of replication-competent adenovirus comprising an adenoviral vector comprising:
(i) an adenoviral genome in which at least a part of the E1 region of the adenoviral genome is deleted; and
(ii) wherein on said adenoviral genome the protein IX gene is in an inverted orientation relative to the direction of transcription of the native protein IX gene at a location where the protein IX gene normally resides,
wherein said inverted protein IX gene is placed under the control of the protein IX natural promoter,
wherein said adenoviral vector comprises a gene of interest,
wherein said inverted protein IX gene and said gene of interest are operably linked to regulatory elements thereby allowing their expression in a host cell, and
wherein said adenoviral vector is prepared in a human embryonic kidney 293 cell which will support the growth of said adenoviral vector.

20. The preparation according to claim 19, wherein all of the E1 region of the adenoviral vector genome is deleted.

21. The preparation according to claim 19, wherein said gene of interest is placed under the control of the cytomegalovirus immediate early promoter (CMV promoter).

22. The preparation according to claim 19, wherein said gene of interest encodes a protein selected from the group consisting of LIGHT, interferon-β, herpes simplex virus thymidine kinase and p53.

23. The preparation according to claim 22, wherein said gene of interest encodes LIGHT.

24. The preparation according to claim 22, wherein said gene of interest encodes interferon-β.

25. The preparation according to claim 24, wherein said interferon-β is human interferon-β.

26. A method of propagating a replication-defective adenoviral particle comprising the steps of:
(a) introducing an adenoviral vector into a host cell, wherein said adenoviral vector comprises an adenoviral genome in which at least a part of the E1 region of the adenoviral genome is deleted;
wherein said protein IX gene on said adenoviral genome is in an inverted orientation relative to the direction of transcription of the native protein IX gene at a location where the protein IX gene normally resides;
wherein said recombinant adenoviral vector comprises a gene of interest;
wherein said inverted protein IX gene and said gene of interest are operably linked to regulatory elements thereby allowing their expression in the host cell;
(b) culturing said host cell comprising said vector for an appropriate period of time and under suitable conditions to allow the production of said viral particle;
(c) recovering said viral particle from said culture; and
(d) optionally, purifying said recovered viral particle.

27. The method according to claim 26, wherein all of the E1 region of the adenoviral genome is deleted.

28. The method according to claim 26, wherein said inverted protein IX gene is placed under the control of the protein IX natural promoter.

29. The method according to claim 26, wherein said gene of interest is placed under the control of the cytomegalovirus immediate early promoter (CMV promoter).

30. The method according to claim 26, wherein said gene of interest encodes a protein selected from the group consisting of LIGHT, interferon-β, herpes simplex virus thymidine kinase and p53.

31. The method according to claim 30, wherein said gene of interest encodes LIGHT.

32. The method according to claim 30, wherein said gene of interest encodes interferon-β.

33. The method according to claim 32, wherein said interferon-β is human interferon-β.

34. The method according to claim 26, wherein said cell is a mammalian cell.

35. The method according to claim 34, wherein said mammalian cell is a human cell.

36. The method according to claim 35, wherein said human cell is a human embryonic kidney 293 cell.

37. The method according to claim 26, wherein there are insufficient overlapping sequences in the right-arm region between said adenoviral vector genome and said host cellular genome to appreciably mediate a recombination event sufficient to result in a replication-competent adenovirus.

38. The method according to claim 37, wherein said overlapping sequences in the right-arm region between said adenoviral genome and said host cellular genome are no more than 280 bp.

39. The method according to claim 38, wherein said overlapping sequences in the right-arm region between said adenoviral genome and said host cellular genome are no more than 264 bp.

40. A system comprising:
(a) a host cell which complements in trans a deficiency in one or more essential gene functions of the E1 region of an adenoviral genome, and
(b) an adenoviral vector comprising: an adenoviral genome in which at least a part of the E1 region of the adenoviral genome is deleted; and
wherein on said adenoviral genome the protein IX gene is in an inverted orientation relative to the direction of transcription of the native protein IX gene at a location where the protein IX gene normally resides,
wherein said inverted protein IX gene is placed under the control of the protein IX natural promoter,
wherein said adenoviral vector comprises a gene of interest,
wherein said inverted protein IX gene and said gene of interest are operably linked to regulatory elements thereby allowing their expression in a host cell,
wherein there are insufficient overlapping sequences between said host cellular genome and said adenoviral genome to appreciably mediate a recombination event sufficient to result in a replication-competent adenovirus.

41. The system according to claim 40, wherein all of the E1 region of the adenoviral genome is deleted.

42. The system according to claim 40, wherein said host cell is a human embryonic kidney 293 cell.

43. The system according to claim 40, wherein said gene of interest is placed under the control of the cytomegalovirus immediate early promoter (CMV promoter).

44. The system according to claim 40, wherein said gene of interest encodes a protein selected from the group consisting of LIGHT, interferon-β, herpes simplex virus thymidine kinase and p53.

45. The system according to claim 44, wherein said gene of interest encodes LIGHT.

46. The system according to claim 44, wherein said gene of interest encodes interferon-β.

47. The system according to claim 46, wherein said interferon-β is a human interferon-β.

48. The system according to claim 40, wherein said overlapping sequences in the right-arm region between said host cellular genome and said adenoviral genome are no more than 280 bp.

49. The system according to claim 48, wherein said overlapping sequences in the right-arm region between said host cellular genome and said adenoviral genome are no more than 264 bp.

50. A pharmaceutical composition comprising an adenoviral vector according to claim 1.

51. A method for treating cancer by in vivo gene therapy comprising the steps of: administering to a subject the adenoviral vector according to claim 1 or a viral particle comprising said adenoviral vector, and allowing said vector or particle to express a gene of interest in said subject, in an amount sufficient to cause cancer regression or inhibition of cancer growth, wherein said gene of interest encodes a protein that causes cancer regression or inhibition of cancer growth.

52. The method according to claim 51, wherein said adenoviral vector or viral particle is administered by a route selected from the group consisting of topical administration, intraocular administration, parenteral administration, intranasal administration, intratracheal administration, intrabronchial administration and subcutaneous administration.

53. The method according to claim 51, wherein said adenoviral vector or viral particle is administered by direct injection at or near a site of a tumor in said subject.

54. The method according to claim 51, wherein said cancer is selected from the group consisting of malignant glioma, melanoma, hemanglioma, leukemia, lymphoma, myeloma, colorectal cancer, non-small cell carcinoma, breast cancer and ovarian cancer.

55. The method according to claim 51, wherein said subject is a human subject.

56. The adenoviral vector according to claim 1, wherein said adenoviral genome is wildtype except for a deletion in at least a part or all of the E1 region and in at least a part of the E3 region of the adenoviral genome.

57. The adenoviral vector according to claim 56, wherein the E3 region of the adenoviral genome is deleted from adenovirus 5 map unit 83.3 through map unit 85.4.

58. The adenoviral vector according to claim 56, wherein said adenoviral genome is wildtype except for a deletion in at least a part of the E1 region, and said deletion is between nucleotide base pairs 353 and 3332.

59. The adenoviral vector according to claim 58, wherein said gene of interest is inserted into said adenoviral genome at the location where the E1 region is deleted between nucleotide base pairs 353 and 3332.

60. The adenoviral vector according to claim 59, wherein said adenoviral vector is AdIFNβ-RIX (also known as H5R9CMVIFNβ).

61. A vaccine composition comprising a recombinant replication-defective adenoviral vector comprising:
(a) an adenoviral genome in which at least a part of the E1 region of the adenoviral genome is deleted; and
(b) wherein on said adenoviral genome the protein IX gene is in an inverted orientation relative to the direction of transcription of the native protein IX gene at a location where the protein IX gene normally resides, wherein said inverted protein IX gene is operably linked to regulatory elements thereby allowing its expression in a host cell.

62. The vaccine composition according to claim 61, wherein the recombinant adenoviral vector comprises a heterologous gene of interest, and which said gene of interest is operably linked to regulatory elements thereby allowing its expression in a host cell.

63. The vaccine composition according to claim 61, wherein all of the E1 region of the adenoviral genome is deleted.

64. The vaccine composition according to claim 61, wherein said inverted protein IX gene is placed under the control of the protein IX natural promoter.

65. The vaccine composition according to claim 62, wherein said gene of interest is placed under the control of the cytomegalovirus immediate early promoter (CMV promoter).

66. The vaccine composition according to claim 62, wherein said gene of interest encodes a protein selected from the group consisting of LIGHT, interferon-β, herpes simplex virus thymidine kinase and p53.

67. The vaccine composition according to claim 66, wherein said gene of interest encodes LIGHT.

68. The vaccine composition according to claim 66, wherein said gene of interest encodes interferon-β.

69. The vaccine composition according to claim 68, wherein said interferon-β is human interferon-β.

70. The vaccine composition according to claim 61, further comprising a therapeutic gene that may have a therapeutic or prophylactic effect in a subject.

71. The vaccine composition according to claim 70, wherein the therapeutic gene is selected from the group consisting of costimulatory proteins of the immune system, interferons, interleukins, growth factors, cytokines, hormones and oncogenes.


SEQUENCE LISTING (23 sequences)

SEQ ID NO: 1 - DNA, 35865 bp, Artificial sequence
  AdIFNBeta-RIX vector (long)
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggagatcagcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcgtcgaggaattcccctctggcacaacaggtagtaggcgacactgttcgtgttgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctggacatatgacgtgagctcagatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagtagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgacttaaaccgcattgggaggggaggaagccttcagggcagaaacctgctggcgcagatccaacagctgctgagaaacgacattaagttcccgggtcaaagaatccaattgtgccaaaagagccgtcaacttgtcatcgcgggcggatgaacgggaagctgcactgcttgcaagcgggctcaggaaagcaaagtcagtcacaatcccgcgggcggtggctgcagcggctgaagcggcggcggaggctgcagtctccaacggcgttccagacacggtctcgtaggtcaaggtagtagagtttgcgggcaggacggggcgaccatcaatgctggagcccatcacattctgacgcaccccggcccatgggggcatgcgcgttgtcaaatatgagctcacaatgcttccatcaaacgagttggtgctcatggcggcggcggctgctgcaaaacagatacaaaactacataagacccccaccttatatattctttcccacccttaagccacgcccacacatttcagtacctcaatctgtatcttcatcgctagagccaaactcagcgcgggtgcaggccagcaccaagtgatcgggcctcagctcctcggtcacatccagcatcacaggctggttcctaatatgtttaccgccacactcgcagggtctgcacctggtgcgggtctcatcgtacctcagcaccttccaaagcttgatccagacatgataagatacattgatgagtttggacaaaccacaactagaatgcagtgaaaaaaatgctttatttgtgaaatttgtgatgctattgctttatttgtaaccattataagctgcaataaacaagttaacttgggccccttgctgcataccacccgcctccaccccataatattatacaaggacacctagtcaaacaaaatgatgcaacttaattttattaggacaaggctggtgggcactggagtagcaccttccacgaccaggagaggcactggggaggggtcacagggatgccacccgggcagacctgaggaagagatggaggtggacgtgtcagcacccggctggggcctgtccctggatgcaggctactctagggcacctgtcccgccttgagctggagggcgaggcctgggctggcttacctaagcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcgnatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgcccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcntgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgaggaacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttctgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagctggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcagctgcttcagcgcgtggctcgttacaacagcggcaacgtgcagaccaacctggaccggctggtgggggatgtgcgcgaggccgtggcgcagctgtgagcgcgcgcagcagcagggcaacctgggctccatggttgcactaaacgccttcctgagtacacagccctgccaacgtgccgcggggacaggaggactacaccaactttgtgagcgcactgcggctaatggtgactgagacaccgcaaagtgaggtgtaccagtctgggccagacttattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcagtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcggcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgtctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggctgccgtgcccggaattgcatcctgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataaccttgactcctaaagtggtattgtacagtgaagatgtagatatagaaaccccagacactcatatnttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttntcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgcctgtataacgctggaaaagtccacccaaagcgtacaggggcccaactcggccgcctgtggactattctgctgcatgtttntccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcacgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgctggagcattaccagcctgctgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttnccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgacgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagncggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagctgctgccatgttgtgttgctaccatgttgttttcatgtgttgctgccatgctcttgtcgccttagatctctctttatgtagtgttgtggtgtctctcttgtcgtgatgtgtgttttgtcctatatattttaatttttaatccaaacccctgtccccgcaggaggcctttggccttctggtaggccgtcattgaaaactgacttaactcgttaaattaaaaaaatgtaaaaaataatggttgagactcagcccaacatcggcagatgaggtggattgagactcagcccaacatcggcagatgaggtggattgagactcaaccccaacattggcagatgaggtgaattagatgaggtggattgagactcatgagggtggtatgagggcccgacgtccacaggtgggagttgtgctttacagtccaacgtgcaggacgcttggcatttgccagagaacaccaagattggcaaattcgcaactggcgccctgtgctcttcacagacggaaaaatgaccaaaatctgattatttttgtaaaacggaaaccgaatgtccgacaaagttcatttgatgacttcccggtaggtctggccctgccgctgggcccgacgccgtccgggaattttacaaacgatttcggacgtctagcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtcttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggctggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 2 - DNA, 24 bp, Artificial sequence
  PCR Sense Primer
taggatccgacgccggccaagaca

SEQ ID NO: 3 - DNA, 24 bp, Artificial sequence
  PCR Antisense Primer
ttaggatccagccagttgggcagc

SEQ ID NO: 4 - DNA, 27 bp, Artificial sequence
  PCR Sense Primer
aagggatccgtcgacgccgccatgagc

SEQ ID NO: 5 - DNA, 36 bp, Artificial sequence
  PCR Antisense Primer
atagaatagcggccgcaaaacccctaaataaagaca

SEQ ID NO: 6 - DNA, 46 bp, Artificial sequence
  PCR  Primer E1BRIX-H3-IVa2 (top)
tcgtacctcagcaccttccaaagcttgctgtctttatttaggggtt

SEQ ID NO: 7 - DNA, 46 bp, Artificial sequence
  PCR Primer RIX-H3-IVa2 (top)
acgcccacacatttcagtacaagcttgctgtctttatttaggggtt

SEQ ID NO: 8 - DNA, 26 bp, Artificial sequence
  PCR Primer IVa2 (bottom)
gtgatcccagaaatatcttcgcccag

SEQ ID NO: 9 - DNA, 46 bp, Artificial sequence
  PCR Primer IVa2-ElbRIX
aacccctaaataaagacagcaagctttggaaggtgctgaggtacga

SEQ ID NO: 10 - DNA, 46 bp, Artificial sequence
  PCR Primer RIX-H3-IVa2 (bottom)
aacccctaaataaagacagcaagcttgtactgaaatgtgtgggcgt

SEQ ID NO: 11 - DNA, 35 bp, Artificial sequence
  PCR Primer RIX (bottom)
gaattcgtcgacttaaaccgcattgggaggggagg

SEQ ID NO: 12 - DNA, 25 bp, Artificial sequence
  hGHpA Forward Primer
agacagcaagcttctgcccgggtgg

SEQ ID NO: 13 - DNA, 25 bp, Artificial sequence
  hGHpA Reverse Primer
ttccaaagcttcacccccctccacc

SEQ ID NO: 14 - DNA, 19 bp, Artificial sequence
  E1 Forward Primer
agatacacccggtggtccc

SEQ ID NO: 15 - DNA, 18 bp, Artificial sequence
  E1 Reverse Primer
cgacgcccaccaactctc

SEQ ID NO: 16 - DNA, 25 bp, Artificial sequence
  E1 Probe
ctgtgccccattaaaccagttgccg

SEQ ID NO: 17 - DNA, 19 bp, Artificial sequence
  E2a Forward Primer
ttgcgtcggtgttggagat

SEQ ID NO: 18 - DNA, 21 bp, Artificial sequence
  E2a Reverse Primer
caaggccaagatcgtgaagaa

SEQ ID NO: 19 - DNA, 20 bp, Artificial sequence
  E2a Probe
tgcaccacatttcggcccca

SEQ ID NO: 20 - DNA, 20 bp, Artificial sequence
  18S Forward Primer
gcctgcggcttaatttgact

SEQ ID NO: 21 - DNA, 20 bp, Artificial sequence
  18S Reverse Primer
caactaagaacggccatgca

SEQ ID NO: 22 - DNA, 33 bp, Artificial sequence
  18S Probe
aaagagctatcaatctgtcaatcctgtccgtgt

SEQ ID NO: 23 - DNA, 35849 bp, Artificial sequence
  AdIFNBeta-RIX vector (short)
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggagatcagcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcgtcgaggaattcccctctggcacaacaggtagtaggcgacactgttcgtgttgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctggacatatgacgtgagctcagatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgacttaaaccgcattgggaggggaggaagccttcagggcagaaacctgctggcgcagatccaacagctgctgagaaacgacattaagttcccgggtcaaagaatccaattgtgccaaaagagccgtcaacttgtcatcgcgggcggatgaacgggaagctgcactgcttgcaagcgggctcaggaaagcaaagtcagtcacaatcccgcgggcggtggctgcagcggctgaagcggcggcggaggctgcagtctccaacggcgttccagacacggtctcgtaggtcaaggtagtagagtttgcgggcaggacggggcgaccatcaatgctggagcccatcacattctgacgcaccccggcccatgggggcatgcgcgttgtcaaatatgagctcacaatgcttccatcaaacgagttggtgctcatggcggcggcggctgctgcaaaacagatacaaaactacataagacccccaccttatatattctttcccacccttaagccacgcccacacatttcagtacctcaatctgtatcttcatcgctagagccaaactcagcgcgggtgcaggccagcaccaagtgatcgggcctcagctcctcggtcacatccagcatcacaggctggttcctaatatgtttaccgccacactcgcagggtctgcacctggtgcgggtctcatcgtacctcagcaccttccaaagcttgatccagacatgataagatacattgatgagtttggacaaaccacaactagaatgcagtgaaaaaaatgctttatttgtgaaatttgtgatgctattgctttatttgtaaccattataagctgcaataaacaagttaacttgggccccttgctccataccacccgcctccaccccataatattatacaaggacacctagtcaaacaaaatgatgcaacttaattttattaggacaaggctggtgggcactggagtagcaccttccacgaccaggagaggcactggggaggggtcacagggatgccacccgggcagacctgaggaagagatggaggtggacgtgtcagcacccggctggggcctgtccctggatgcaggctactctagggcacctgtcccgccttgagctggagggcgaggcctgggctggcttacctaagcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcgnatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgaggaacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcagtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttntcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttntccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttnccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgacgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagncggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagctgctgccatgttgtgttgctaccatgttgttttcatgtgttgctgccatgctcttgtcgccttagatctctctttatgtagtgttgtggtgtctctcttgtcgtgatgtgtgttttgtcctatatattttaatttttaatccaaacccctgtccccgcaggaggcctttggccttctggtaggccgtcattgaaaactgacttaactcgttaaattaaaaaaatgtaaaaaataatggttgagactcagcccaacatcggcagatgaggtggattgagactcagcccaacatcggcagatgaggtggattgagactcaaccccaacattggcagatgaggtgaattagatgaggtggattgagactcatgagggtggtatgagggcccgacgtccacaggtgggagttgtgctttacagtccaacgtgcaggacgcttggcatttgccagagaacaccaagattggcaaattcgcaactggcgccctgtgctcttcacagacggaaaaatgaccaaaatctgattatttttgtaaaacggaaaccgaatgtccgacaaagttcatttgatgacttcccggtaggtctggccctgccgctgggcccgacgccgtccgggaattttacaaacgatttcggacgtctagcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgttttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg